(19) United States
(12) Patent Application Publication
(10) Pub. No.: US 2016/0091498 A1
(43) Pub. Date: Mar. 31, 2016

(54) METHODS FOR THE TREATMENT OF DISORDERS RELATED TO PHOSPHORYLATION OF HISTONES

(71) Applicant: Nupam P. Mahajan, Tampa, FL (US)

(72) Inventor: Nupam P. Mahajan, Tampa, FL (US)

(21) Appl. No.: 14/860,405

(22) Filed: Sep. 21, 2015

Related U.S. Application Data
(60) Provisional application No. 62/054,209, filed on Sep. 23, 2014.

(51) Int. Cl. G01N 33/68; C07K 16/18

(52) U.S. Cl. CPC G01N 33/6875; C07K 16/18; G01N 2800/52; G01N 2333/47

(57) ABSTRACT

Methods for disease diagnosis, prognosis and therapy selection. Compositions for use in these methods and selected therapies for treatment are also disclosed.

### CROSS-REFERENCE TO RELATED APPLICATION

This application claims priority under 35 U.S.C. §119(e) to U.S. Provisional Application No. 62/054,209, filed Sep. 23, 2014, the content of which is incorporated herein by reference in its entirety.

### FIELD OF THE DISCLOSURE

The disclosure is generally related to methods for detecting phosphorylated histones and their use in companion diagnostics. Also disclosed are methods of disease diagnosis, prognosis and therapy selection based on such detection and determination.

### BACKGROUND

Histones are abundant and highly essential eukaryotic proteins that are basic in nature. Two molecules of each of the four core histones, H2A, H2B, H3 and H4, constitute the histone octamer, around which 147 base pairs of DNA are wrapped to form the nucleosome core particle. The core particle is the fundamental repeating unit of eukaryotic chromatin. A linker histone, also known as histone H1, is present in higher eukaryotes and seals two full turns of the DNA to form the complete nucleosome. The major function of nucleosome was appreciated early—this nucleosomal structure is repeated until the entire genomic DNA is packaged into chromatin fibers. The chromatin fibers undergo further compaction to form chromosomes, the basic units of genetic information in all living eukaryotes. In last decade or so it became apparent that histones and chromatin structure regulate access to the information contained within the DNA. And this information plays a crucial role in majority of cellular and metabolic processes, e.g., transcription, replication, recombination and DNA damage and repair. This truly has opened the door for the deeper understanding of how histone modification and subsequent changes in chromatin regulates normal human physiology. But the most critical issue is how this process is involved in various diseases, e.g., cancer, diabetes and aging.

It is becoming clear that post-translational modifications of histones are important. So far, the core histones have been shown to be phosphorylated, acetylated, methylated, sumoylated, ribosylated and ubiquitylated at various amino acid residues, forming a ‘histone code’ or ‘epigenetic code’. The histone code suggests that histone modifications not only alter the affinity of histones for DNA but importantly act as recognition or binding sites for various factors or proteins to assemble at the site of modification. This results in relay of information that leads to initiation or suppression of specific cellular event or process. Interestingly, the epigenetic code also results in crosstalk between the different modifications, e.g., phosphorylation, methylation, acetylation and ubiquitination.

Histone modifications are the epigenetic changes. It is now known that in addition to genetic defects, epigenetic defects can also result in disease. Epigenetics is also thought to play a major role in the pathogenesis of common, multifactorial disorders. For example, there is evidence suggesting that the primary (idiopathic) disorders like schizophrenia and bipolar disorder are epigenetic defects rather than genetic defects. Epigenetic factors have also been shown to be involved in aging, in rare monogenic disorders like fragile-X mental retardation, and in lymphomas.

### SUMMARY

Applicant provides herein a method for identifying or selecting a cancer patient having a wildtype BRAF genotype or patients with mutant BRAF having developed resistance against BRAF inhibitors such as Vemurafenib or Dabrafenib for a therapy, wherein the therapy comprises, or alternatively consisting essentially of, or yet further consisting of, a WEE1 inhibitor, the method comprising, or alternatively consisting essentially of, or yet further consisting of, detecting phosphorylation of a histone H2B protein at the Tyr37 residue in a sample isolated from the patient, wherein phosphorylation of the H2B at Tyr37 residue selects or identifies the patient for the therapy and absence of phosphorylation of the histone H2B protein at the Tyr37 residue does not identify or select the patient for the therapy. In a further aspect, the BRAF genotype of the patient may be unknown or unverified, and then the method can further comprise determining the BRAF genotype of the patient prior to detecting the phosphorylation of histone H2B in the patient. The method can, in one aspect, further comprise, or alternatively consist essentially of, or yet further consist of, administering an effective amount the WEE1 inhibitor to the cancer patient, e.g., MK-1775 or AZD-1775). Such therapies are known in the art and are described herein.

Applicant provides methods for selecting a cancer patient having a wildtype BRAF genotype for a therapy, wherein the therapy comprises, or alternatively consisting essentially of, or yet further consisting of, a WEE1 inhibitor (e.g., MK-1775 or AZD-1775), by determing the expression level of the WEE1 RNA or protein and IDH2 RNA or protein in a sample isolated from the patient, wherein a) overexpression of WEE1 RNA or protein and b) underexpression of IDH2 RNA or protein in the sample as compared to a control for the WEE1 RNA or protein expression level and a control for the IDH2 RNA or protein, respectively, selects the cancer patient for the therapy and neither a) nor b) does not select the patient for the therapy. The method can, in one aspect, further comprise, or alternatively consist essentially of, or yet further consist of, administering an effective amount the WEE1 inhibitor to the cancer patient. Such therapies are known in the art and are described herein.

Also provided herein is a method for identifying or selecting a melanoma or brain cancer patient for a therapy comprising, or alternatively consisting essentially of, or yet further consisting of, administration of an WEE1 inhibitor (e.g., MK-1775 or AZD-1775), by detecting phosphorylation of a histone H2B protein at the Tyr37 residue in a sample isolated from the patient, wherein phosphorylation of the Tyr37 residue selects or identifies the patient for the therapy and absence of phosphorylation of the histone H2B protein at the Tyr37 residue does not identify or select the patient for the therapy. In a further aspect, an effective amount of the therapy is administered to the patient identified or selected for the therapy. Such therapies are known in the art and are described herein. Suitable samples comprise melanoma cells or tumor samples.

Suitable cancer patients for this method include for example, a cancer patient suffers from brain cancer (glioblastoma multiforme, breast cancer, melanoma, lung cancer and prostate cancer).

Samples for use in the method comprise, or alternatively consist essentially of, or yet further consist of, one or more of a cancer cell or blood sample.

Any appropriate method for determining the expression level of the WEE1 protein can be used. Non-limiting examples of such include a method comprising determining the amount of mRNA encoding the WEE1 protein in the sample by quantitative RT-PCR or a method comprising immunohistochemistry. In one aspect, the expression level of the WEE1 protein is determined by determining Y37-H2B phosphorylation in the sample. In one aspect, phosphorylation of Y37-H2B is determined by contacting the sample with an antibody that specifically recognizes and binds the phosphorylated Y37-H2B in the sample if it exists. In another aspect, the antibody is a monoclonal antibody.

Yet further, the expression level of the IDH2 RNA is determined by a method comprising determining the amount of mRNA encoding the IDH2 protein in the sample or by a method comprising quantitative RT-PCR. In one aspect the immunohistochemical method comprises the use of an WEE1 and/or IDH2 specific antibody or 5-hmC (5-Hydroxymethylcytosine) by immunohistochemistry. In one aspect, the antibody is not a polyclonal or naturally-occurring antibody.

Also provided herein is a method for identifying or selecting a castration resistant prostate cancer (CRPC) patient for a therapy comprising, or alternatively consisting essentially of, or yet furher consisting of, administration of an ACK1 inhibitor, by detecting phosphorylation of a histone H4 protein at the Tyr88 residue in a sample isolated from the patient, wherein phosphorylation of the Tyr88 residue selects or identifies the patient for the therapy and absence of phosphorylation of the histone H4 protein at the Tyr88 residue does not identify or select the patient for the therapy. In a further aspect, an effective amount of the therapy is administered to the patient identified or selected for the therapy. Such therapies are known in the art and are described herein. Suitable samples comprise CRPC cells or tumor samples, biopsies or blood samples.

Any suitable method for detecting the phosphorylation can be used. As a non-limiting example, the detecting comprising contacting the sample with an isolated antibody that specifically recognizes SEQ ID NO: 1 (KRISGLIpYEETRGVL), wherein the Y(Tyr)8 residue is phosphorylated. In one aspect, the antibody is not a polyclonal antibody or an isolated naturally occurring antibody.

Yet further provided is a method for identifying or selecting a subject in need thereof for a therapy comprising an ACK1 inhibitor, comprising determing the level of phosphorylation of a histone H3 protein at the Tyr99 residue in a sample isolated from the subject, and identifying or selecting the subject for the therapy if phosphorylation of the Tyr99 residue is detected and not selecting the patient for the therpy if the phosphorylation of the Tyr99 is not detected. Non-limiting examples of subjects in need of such treatment include a patient suffering from a disorder selected from infantile-onset epilepsy, cognitive regression, obsessive-compulsive disorder (OCD), depression, substance dependence, and cocaine dependence. ACK1 inhibitors are known in the art and described herein. In one aspect, the method further comprises administering to the subject identified or selected for the therapy an effective amount of the ACK1 inhibitor. Suitable samples for the method include tissues, cell, biopsies, saliva, semen, cheek or mouth swab or blood samples.

Any appropriate method for determing the level of phosphorylation of a histone H3 protein at the Tyr99 residue can be used, for example by contacting the sample with an isolated antibody that specifically recognizes a histone H3 protein comprising a phosphorylated Tyr99 residue, e.g., an isolated antibody specifically recognizes SEQ ID NO: 2 (ALQEACEApYLVGLFED), wherein the Y(Tyr)9 residue is phosphorylated. In one embodiment, such an antibody is not a polyclonal antibody or an isolated naturally occurring antibody.

Compositions for use in the above methods are further provided.

A composition comprising an antibody that specifically recognizes SEQ ID NO: 1 (KRISGLIpYEETRGVL), wherein the Y(Tyr)8 residue is phosphorylated for use in a method for identifying or selecting a castration resistant prostate cancer (CRPC) patient for a therapy comprising an ACK1 inhibitor is provided.

A composition comprising a probe and/or antibody for determining the expression level of Histone H2B Tyr37-phosphorlation and WEE1 RNA or protein in sample for use in a method for selecting a cancer patient having a wildtype BRAF genotype and patients who have developed anti-BRAF inhibitor resistance for a therapy comprising a WEE1 inhibitor is provided.

A composition comprising a probe and/or antibody for determining the loss or decrease in expression level of IDH2 RNA or protein for use in a method for selecting a cancer patient having a wildtype BRAF genotype and patients who hve developed resistance for BRAF inhibitor for a therapy comprising a WEE1 inhibitor is provided.

A composition comprising an antibody specifically recognizes SEQ ID NO: 2 (ALQEACEApYLVGLFED), wherein the Y(Tyr)9 residue is phosphorylated for identifying or selecting a therapy comprising an ACK1 inhibitor for a subject suffereing from a disorder selected from infantile-onset epilepsy, cognitive regression, obsessive-compulsive disorder (OCD), depression, substance dependence, and cocaine dependence also is provided.

Kits containing one or more of the above compositions and instructions for use are further provided herein.

### BRIEF DESCRIPTION OF THE DRAWINGS

Provided as embodiments of this disclosure are drawings which illustrate by exemplification only, and not limitation.

FIG. 1 shows suppression of IDH2 expression in melanomas. qRT-PCR of normal skin (6 samples) and melanoma RNA (patient #1-14) in triplicates. Relative expression of IDH2 is shown. NT, BRaf status unknown.

FIGS. 2A-2E show validation of pY99-H3 antibody by dot blot analysis of peptides. (FIG. 2A) Peptides spanning Tyr99 site, ALQEACEApYLVGLFED and identical but unmodified peptide and a point mutation containing peptide ALQEACEAFLVGLFED were spotted on nitrocellulose membrane followed by immunoblotting with pY99-H3 antibody (top panel). The pY99-H3 antibody specifically recognized phosphorylated peptide but failed to recognize unphosphorylated or Y99F mutant peptide. Prior to probing the dot blot, the pY99-H3 antibody was pre-incubated with the phosphopeptide ALQEACEApYLVGLFED (bottom panel). The phosphopeptide competed with pY99-H3 antibody for binding to H3 phosphopeptide that has been spotted on the filter. (FIG. 2B) Peptide spanning Tyr99 site, ALQEACEApYLVGLFED were spotted on nitrocellulose membrane in increasing concentration followed by immunoblotting with pY99-H3 antibody (top panel). Prior to probing the dot blot, the pY99-H3 antibody was pre-incubated with the phosphopeptide ALQEACEApYLVGLFED (bottom panel). (FIG. 2C) Peptide spanning Y99 site, ALQEACEApYLVGLFED and derived from all the four core histones, H2A, H2B, H3 and H4 were spotted on nitrocellulose membrane followed by immunoblotting with pY99-H3 antibody. The pY99-H3 antibody specifically recognized Y99 phosphorylated H3 peptide but failed to recognize unphosphorylated H3 or peptides derived from other core histones. (FIG. 2D) The pY99-H3 antibody characterization. The MODified Histone Peptide Array (Acitve Motif) containing 384 unique histone modification combinations, was immunoblotted with pY99-H3 (top panel) or H3K4me3 antibodies (as control). The pY99-H3 antibody did not recognize any of the 59 acetylation, methylation, phosphorylation, and citrullination modifications on histones H2A, H2B, H3 and H4. (FIG. 2E) MCF-7 cells were treated with heregulin ligand for indicated time points. Equal amounts of lysates were immunoblotted with pY99-H3 antibody (top panel).

FIGS. 3A-3E show ACK1 phosphoryltes H3 at Tyr99 residue. (FIG. 3A) Alignment of H3 protein sequences indicates that tyrosine residue at 99 is invariant from human to yeast. (FIG. 3B) HEK293 cells co-expressing Myc-tagged ACK1 (WT) or KD mutant ACK1 and FLAG-tagged H3 or Y99F mutant H2B were serum-starved (24 h) and lysates were immunoprecipitated with pY99-H3 antibodies followed by immunoblotting with FLAG antibody (top panel). (FIG. 3C) HEK293 cells were treated with EGF ligand for indicated time points. Equal amounts of lysates were immunoprecipitated pY99-H3 antibodies followed by with immunoblotting with H3 antibody (top panel). (FIG. 3D) HEK293 cells were treated with ACK1 inhibitor, AIM-100 (5 uM overnight) and/or EGF ligand 20 min. Equal amounts of lysates were immunoprecipitated pY99-H3 antibodies followed by with immunoblotting with H3 antibody (top panel). ACK1 inhibition suppressed histone H3 Tyr99-phosphorylation. (FIG. 3E) HEK293 cells were treated with increasing concentrations of AIM-100 (1, 2, 3 and 5 μM). Equal amounts of lysates were immunoprecipitated pY99-H3 antibodies followed by with immunoblotting with H3 antibody (top panel).

FIGS. 4A-4E show that loss of ACK1 results in loss of SLC6A4 expression. (FIG. 4A) Total RNA was prepared from the brains of WT and KO mice followed by quantitative RT-PCR using ACK1 and actin primers. The ratio of ACK1 to actin is shown. (FIG. 4B) Total RNA was prepared from the brains of WT and KO mice followed by quantitative RT-PCR using SLC6A4 and actin primers. The ratio of SLC6A4 to actin is shown. (FIG. 4C) Lysates prepared from WT and KO brains were subjected to immunoblotting with indicated antibodies. (FIG. 4D) Lysates prepared from WT and KO pancreas were subjected to immunoblotting with indicated antibodies. (FIG. 4E) Mouse embryo fibroblast cell lines were generated from ACK1 WT and KO mice. The lysates prepared from WT cell lines were subjected to immunoblotting with indicated antibodies.

FIGS. 5A-5D show ACK1 deposits pY99-H3 epigenetic marks in SLC6A4 promoter and intron 2. (FIG. 5A) Lysates prepared from JAR cells treated with EGF ligand were subjected to immunoblotting with indicated antibodies. (FIG. 5B) Lysates prepared from JAR cells treated with EGF or AIM-100 were subjected to immunoblotting with indicated antibodies. (FIG. 5C) Total RNA was prepared from JAR cells untreated or treated with EGF ligand followed by quantitative RT-PCR using SLC6A4 and actin primers. The ratio of SLC6A4 to actin is shown. (FIG. 5D) JAR cells were treated with EGF ligand and ChIP was performed using pY99-H3 antibodies followed by quantitative PCR using primers corresponding to intron 2 (VNTR).

FIGS. 6A-6E show that ACK1 kinase activity is required to maintain androgen-independent AR protein levels. (FIG. 6A) Androgen starved LAPC4 cells were treated with DHT (10 nM, 16 Hrs), AIM-100, DZ1-067, Enzalutamide or PLX4032 (7 uM, 36 Hr) and AR, pACK1 and Actin levels were determined by immunoblotting (IB). The relative level of AR is shown below. (FIG. 6B) Androgen deprived LNCaP and VCaP cell were untreated or treated with 2.5, 5 and 10 uM of AIM-100 and lysates were immunoblotted. The quantitation of AR expression is shown. (FIG. 6C) Androgen deprived LNCaP and VCaP cell were untreated or treated with 2.5, 5 and 10 uM of DZ 1-067 and lysates were immunoblotted. The quantitation of AR expression is shown. (FIG. 6D) LNCaP cells were transfected with control and ACK1 siRNAs followed by immunoblotting. (FIG. 6E) LNCaP cells treated with AIM-100 (7 uM, 16 hr) or MG132 (10 uM, 6 hr) were immunoblotted with AR antibodies

FIGS. 7A-7D show that inhibition of ACK1 kinase activity suppresses AR transcription. (FIG. 7A) VCaP and (FIG. 7B) LNCaP cells were grown in absence of androgen and were overnight treated with DZ1-067 (7 uM in VCaP, 2.5 & 5 uM in LnCaP), AIM-100 (7 uM), PLX4032 (7 uM), Casodex, Enzalutamide (10 uM) and DHT (10 nM, 3 Hr). Total RNA was isolated followed by qPCR with AR primers. VCaP, *p=0.022, **p=0.018; LNCaP, *p=0.042, **p=0.047, ***p=0.029. (FIG. 7C) VCaP and (FIG. 7D) LNCaP cells were grown in absence of androgen and were overnight treated with DZ1-067 (7 uM in LAPC4, 2.5 & 5 uM in LnCaP), AIM-100 (7 uM), PLX4032 (7 uM), Casodex, Enzalutamide (10 uM) and DHT (10 nM, 3 Hr). Total RNA was isolated followed by qPCR with PSA primers.

FIGS. 8A and 8B show cell proliferation assay. (FIG. 8A) LNCaP and (FIG. 8B) VCaP cells were grown in charcoal stripped media and treated with 1, 2.5, 5 and 10 uM of inhibitors (36 hrs) and number of viable cells were counted by trypan blue exclusion assay

FIGS. 9A-9E show ACK1 phosphorylates histone H4 at Tyrosine 88. (FIG. 9A) peptide were spotted followed by immunoblotting with indicated antibodies. (FIG. 9B) Equimolar amounts of purified ACK1 and H4 proteins were incubated in the presence of AIM-100 (100 nM) and reaction subjected to immunoblotting with pTyr antibodies. (FIG. 9C) In vitro kinase assay performed using purified ACK1 and H4, followed by immunoblotting with pY88-H4 antibodies. (FIG. 9D) H4 Y88-phosphorylation in vivo. LNCaP cells were treated with DZ1-067, AIM-100 or Enzalutamide (7 uM, 16 hrs). The nuclear lysates were immunoprecipitated with pY88-H4 antibodies followed by immunoblotting with H4. Lower panel is input lysate. (FIG. 9E) LNCaP cells were treated with Crizotinib (1 uM, 16 hrs). The cell lysates were immunoprecipitated with pY88-H4 antibodies followed by immunoblotting with H4 (upper panel). Lower panel is input lysate immunblotted with H4 antibodies.

FIGS. 10A-10C show H4 Y88-phosphorylation occurs within and downstream of AR gene. (FIG. 10A) The human AR gene and two pY88-H4 binding sites are shown. (FIG. 10B) VCaP and (FIG. 10C) LNCaP cells treated with ACK1 inhibitor; ChIP was performed followed by qPCR using primers corresponding to promoter, intron 2, 3′UTR or control region. VCaP: *p<0.05, **p<0.05; LNCaP: *p<0.05, **p<0.05

FIG. 11 shows validation of ACK1 specific gRNA construct. NT: PCR product from non targeted cells showing specific product of ˜681 bp. Cas9 only: Control line from only Cas9 plasmid transfection showing no-off target cutting of Cas9 in the absence of gRNA. +ve control: Positive control gDNA cut with Cas9. Un-cut control: PCR product from transfected cells showing the 681 bp band. Next 3 lanes are different gRNAs for ACK1

FIG. 12 shows that specific interaction of the pY88-H4 marks with the chromatin remodeling protein WDR5. Peptide pull down assays reveal increased binding of WDR5 to the phosphorylated H4 peptide compared to the unphosphorylated H4 peptide.

FIGS. 13A-13D show that recruitment of AR and deposition of H3K4me3 epigenetic marks within intron 2 of AR gene. LNCaP cells treated with AIM-100 and ChIP was performed using AR, H3K4me3 or IgG antibodies followed by qPCR using primers corresponding to intron 2, or control region. *p<0.05, **p<0.05.

FIG. 14 shows pY88-H4 and AR staining of human prostate samples. Tissue Micro Array (TMA) sections representing different prostate cancer stages stained with pY88-H4 and AR Antibodies.

Some or all of the figures are schematic representations for exemplification; hence, they do not necessarily depict the actual relative sizes or locations of the elements shown. The figures are presented for the purpose of illustrating one or more embodiments with the explicit understanding that they will not be used to limit the scope or the meaning of the claims that follow below.

### DETAILED DESCRIPTION

### I. Definitions

The term “phosphospecific probe” refers to a composition that specifically binds a target antigen in its phosphorylated state but does not specifically bind the antigen when it is not phosphorylated. The probe is preferably an antibody (i.e., a phosphospecific antibody).

The term “antibody” refers to a polyclonal, monoclonal, recombinant, or synthetic immunoglobulin molecule that specifically binds a target antigen. In one aspect, monoclonal antibodies are excluded. In another embodiment, polyclonal antibodies or other naturally occurring antibodies are excluded. The term includes intact immunoglobulin molecules, fragments or polymers of those immunoglobulin molecules, chimeric antibodies containing sequences from more than one species, class, or subclass of immunoglobulin, and human or humanized versions of immunoglobulin molecules or fragments thereof containing a least the idiotype of an immunoglobulin that specifically binds the target antigen.

The term “idiotype” refers to the portion of an immunoglobulin molecule that confers the molecule's ability to bind an antigen. The idiotype of an antibody is determined by the complementarity determining regions (CDRs) of the immunoglobulin variable domains (V_{L} and V_{H}).

The term “peptide” or “polypeptide” can be used to refer to a natural or synthetic molecule comprising two or more amino acids linked by the carboxyl group of one amino acid to the alpha amino group of another. The peptide is not limited by length; thus “peptide” can include polypeptides and proteins.

The term “peptidomimetic” refers to a mimetic of a peptide which includes some alteration of the normal peptide chemistry. Peptidomimetics typically enhance some property of the original peptide, such as increase stability, increased efficacy, enhanced delivery, increased half life, etc.

The term “aptamer” refers to an oligonucleic acid molecule that specifically binds to a target molecule.

As used herein, the term “small molecule” refers to a compound having a molecular weight of less than 1000 Daltons, and typically between 300 and 700 Daltons. The term may include monomers or primary metabolites, secondary metabolites, a biological amine, a steroid, or synthetic or natural, non-peptide biological molecule(s). In the context of targeted imaging probes that are small molecules, the small molecule can specifically bind the molecular or cellular target.

The term “specifically recognizes” or “specifically binds” refers to the recognition or binding of a molecule to a target molecule, such as an antibody to its cognate antigen, while not significantly binding to other molecules. Preferably, a molecule “specifically binds” to a target molecule with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with the target molecule.

The term “neoplasm” refers to a cell undergoing abnormal cell proliferation. The growth of neoplastic cells exceeds and is not coordinated with that of the normal tissues around it. The growth typically persists in the same excessive manner even after cessation of the growth or other stimuli, and typically causes formation of a tumor. Neoplasms may be benign, premalignant, or malignant.

The term “cancer” or “malignant neoplasm” refers to a cell that displays uncontrolled growth, invasion upon adjacent tissues, and often metastasizes to other locations of the body.

The term “subject” or “patient” refers to any individual who is the target of administration. The subject can be a vertebrate, for example, a mammal. Thus, the subject can be a human. The subject can be domesticated, agricultural, or zoo- or circus-maintained animals. Domesticated animals include, for example, mice, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses. The term does not denote a particular age or sex.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. The term “isolated” is also used herein to refer to cells or tissues that are isolated from other cells or tissues and is meant to encompass both cultured and engineered cells or tissues.

It is to be inferred without explicit recitation and unless otherwise intended, that when the present invention relates to a polypeptide, protein, polynucleotide or antibody, an equivalent or a biologically equivalent of such is intended within the scope of this invention. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. In one aspect, a biological equivalent of an antibody means one having the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

When a genetic marker, e.g., overexpression of WEE1, is used as a basis for selecting a patient for a treatment described herein, the genetic marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker in a clinical setting is a clear indication that this parameter was used as a basis for initiating, continuing, adjusting and/or ceasing administration of the treatments described herein.

WEE1 gene or polynucleotide encodes a nuclear protein, which is a tyrosine kinase, belonging to the Ser/Thr family of protein kinases. The gene and the protein it encodes have been characterized. The amino acid of the human sequence is deposited at NP_001137448, and the mouse amino acid sequence NP_033542. The sequence of the mRNA encoding the human protein is available at GenBank NM_001143976 and the mouse mRNA is available at GenBank NM_009516. Monoclonal antibodies that specifically recognize and hind the proteins, for immunohistochemical analysis, can be purchased from Santa Cruze Biotechnology (sc-5285) or generated using methods known in the art.

Isocitrate dehydrogenase is an enzyme that is encoded in humans the IDH2 gene. The amino acid sequence for the human protein is available at GenBank NP_002159 and the mouse protein is available at NP_766599. The mRNA encoding the proteins are available at GenBank NM_002168 (human) and NM_173011 (mouse). Antibodies that specifically recognize and bind the protein can be made using well known methods or purchased from abeam (ab131263).

As used herein, “BRAF” intends the gene that encodes a protein called B-Raf or in some aspects, v-Raf the murine homolog. The amino acid sequence for the human protein is available at GenBank NP_00004324 and the mouse protein is available at NP_647455. The mRNA encoding the proteins are available at GenBank NM_004333 (human) and NM_139294 (mouse). Antibodies that specifically recognize and bind the protein can be made using well known methods or purchased from abeam or Santa Cruz Biotechnology (sc-5284).

ACK1 (also known as TNK2) gene or polynucleotide encodes a cytoplasmic protein that translocates to nucleus (nuclear protein), which is a tyrosine kinase, belonging to the non-receptor tyrosine kinase family of protein kinases. The gene and the protein it encodes have been characterized. The amino acid of the human sequence is deposited at NP_005772, and the mouse amino acid sequence NP_058068. The sequence of the mRNA encoding the human protein is available at GenBank NM_005781 and the mouse mRNA is available at GenBank NM_016788. Monoclonal antibodies that specifically recognize and hind the proteins, for immunohistochemical analysis, can be purchased from Santa Cruz Biotechnology (sc-28336) or generated using methods known in the art.

“Cancer” is a known medically as a malignant neoplasm, is a broad group of diseases involving unregulated cell growth. In cancer, cells divide and grow uncontrollably and in one aspect, forming malignant tumors, and invade nearby parts of the body. Non-limiting examples include colon cancer, colorectal cancer, gastric cancer, esophogeal cancer, head and neck cancer, breast cancer, lung cancer, stomach cancer, liver cancer, gall bladder cancer, or pancreatic cancer or leukemia, prostate and brain cancer.

A “composition” as used herein, intends an active agent, such as a compound as disclosed herein and a carrier, inert or active. The carrier can be, without limitation, solid such as a biotin, a bead or a resin, or liquid, such as phosphate buffered saline.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response.

### II. Detailed Description

It is discovered herein that certain tyrosine residues of various histone proteins, which are not known to be subject to epigenetic modifications, can be phosphorylated in cells. Such amino acid residues include H2B Tyr37, H4 Tyr88 and Tyr 51 and H3 Tyr99. The present disclosure further provides experimental data to reveal the kinases that can phosphorylate these residues, the impact of such phosphorylation to a cell including gene expression changes, and their clinical implications.

### Diagnostic Methods

The present disclosure builds upon the discovery of a newly discovered phosphorylation event, tyrosine 37 in histone H2B (pY37-H2B) mediated by WEE1 tyrosine kinase. The identification was facilitated and confirmed by newly generated pY37-H2B specific antibodies. Such phosphorylation can occur in certain cancer cells, including brain cancer cells (e.g., GBM), breast cancer cells (e.g., triple negative breast cancer), prostate cancer, pancreatic cancer, melanoma and lung cancer cells that display activation of WEE1 or ACK1. Therefore, by detecting the Y88-H4 phosphorylation, a cell can be assayed for its WEE1 or ACK1 kinase activity and assessed for its status in carcinogenesis, as well as its suitability for a treatment targeting these kinases.

It is shown that the Tyr88 residue of H4 can be phosphorylated by the WEE1 or Ack1 kinase. Such phosphorylation can occur in certain cancer cells, including brain cancer cells (e.g., GBM), breast cancer cells (e.g., triple negative breast cancer), prostate cancer, pancreatic cancer, melanoma and lung cancer cells that display activation of WEE1 or Ack1. Therefore, by detecting the Y88-H4 phosphorylation, a cell can be assayed for its WEE1 or Ack1 kinase activity and assessed for its status in carcinogenesis, as well as its suitability for a treatment targeting these kinases.

In one aspect, the methods as disclosed herein utilized antibodies that specifically recognize and bind the proteins of interest. Antibodies, raised against polypeptides described above for use in these methods are described below.

### 1. Antibodies

In preferred embodiments, the phosphospecific probes are antibodies. Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Preferred CDRs are the CDRs in the example phosphospecific antibodies described in the Examples.

Antibodies for use in the disclosed compositions and methods can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. Fab is the fragment of an antibody that contains a monovalent antigen-binding fragment of an antibody molecule. A Fab fragment can be produced by digestion of whole antibody with the enzyme papain to yield an intact light chain and a portion of one heavy chain. Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region. (Fab′)₂ is the fragment of an antibody that can be obtained by treating whole antibody with the enzyme pepsin without subsequent reduction. F(ab′)₂ is a dimer of two Fab′ fragments held together by two disulfide bonds. Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

Techniques can also be adapted for the production of single-chain antibodies specific for the cellular targets. Single chain antibody (“SCA”), defined as a genetically engineered molecule containing the variable region of the light chain (V_{L}), the variable region of the heavy chain (V_{H}), linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the V_{H} and V_{L} domains that enables the sFv to form the desired structure for antigen binding. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two V_{H} and two V_{L} regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies. Preferably, if the antibody is to be administered to humans, the antibody is a human antibody or is a “humanized” antibody derived from a non-human animal.

This disclosure also provides recombinant polynucleotides encoding the antibodies or fragments thereof, as described above. The polynucleotides can be chemically synthesized or recombinantly produced in host cell systems using methods known in the art.

### 2. Peptides

In some embodiments, the phosphospecific probe can be a peptide. In some embodiments, the peptide can contain the idiotype of an antibody, such as those described above. In other embodiments, the peptide can be identified by screening a library of peptides against the phosphorylated histone.

### 3. Peptidomimetics

In some embodiments, the phosphospecific probe can be a peptidomimetic. In some embodiments, the peptidomimetic can mimic the idiotype of an antibody, such as those described above. In other embodiments, the peptidomimetic can be identified by screening a library of peptidomimetic against the phosphorylated histone.

A peptidomimetic is a small protein-like chain designed to mimic a peptide. They typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. This can have a role in the development of drug-like compounds from existing peptides. These modifications involve changes to the peptide that will not occur naturally (such as altered backbones and the incorporation of nonnatural amino acids).

Peptidomimetics can have a non-amino acid residue with non-amide linkages at a given position. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

### 4. Aptamers

In some embodiments, the phosphospecific probe is an aptamer. Aptamers are single-stranded RNA or DNA oligonucleotides 15 to 60 base in length that bind with high affinity to specific molecular targets. Most aptamers to proteins bind with Kds (equilibrium constant) in the range of 1 pM to 1 nM, similar to monoclonal antibodies. These nucleic acid ligands bind to nucleic acid, proteins, small organic compounds, and even entire organisms.

Aptamers can be selected by incubating the target molecule in a large (e.g., 1010 to 1020) pool of oligonucleotide (usually 40 to 60mers). The large pool size of the oligonucleotide ensures the selection and isolation of the specific aptamer. Aptamers can distinguish between closely related but non-identical members of a protein family, or between different functional or conformational states of the same protein. The protocol called systematic evolution of ligands by exponential enrichment (SELEX) is generally used with modification and variations for the selection of specific aptamers. Using this process, it is possible to develop new aptamers in as little as two weeks.

### Phosphorylated Histone or Histone Fragments

The present disclosure provides new phosphorylation sites of histones H2B, H3 and H4. Accordingly, one embodiment provides isolated H2B, H3 or H4 proteins or protein fragments that contain one or more of these sites.

In one embodiment, provided is an isolated polypeptide that includes an amino acid sequence as shown below or one that has at least about 80%, 85%, 90%, 95%, 98% or 99% sequence identity to the sequence shown:

| | |
|---|---|
| KRSRKESYSVYVYKVL | (SEQ ID NO: 3) |
| KRSRKESpYSVYVYKVL | (SEQ ID NO: 4) |
| TVTAMDVVYALKRQGRT | (SEQ ID NO: 5) |
| TVTAMDVVpYALKRQGRT | (SEQ ID NO: 6) |
| KRISGLIYEETRGVL | (SEQ ID NO: 7) |
| KRISGLIpYEETRGVL | (SEQ ID NO: 1) |
| ALQEACEAYLVGLFED | (SEQ ID NO: 8) |
| ALQEACEApYLVGLFED, | (SEQ ID NO: 2) |

wherein a lowercase letter “p” indicates that the amino acid following it is phosphorylated.

The unphosphorylated peptides here can be used as substrate to measure the activities of kinases responsible for phosphorylation of one of these sites. The phosphorylated one, on the other hand, can be used to generate or verify antibodies or other types of probes that specifically recognize or bind them. In some aspects, the peptides do not include (e.g., are shorter than) the entire histone protein.

### Kits

One or more of the compositions described herein can be assembled in kits. Printed instructions, either as inserts or as labels, indicating quantities of the components to be administered, guidelines for administration, and/or guidelines for mixing the components, can also be included in the kit. Kits of the disclosure can optionally include pharmaceutically acceptable carriers and/or diluents.

The disclosed kit can contain, for example, phosphospecific probes, such as antibodies, that specifically bind one, two, three, or more of H2B-Tyr37, H4-Tyr88, H4-Tyr51, and H3-Tyr99.

In some aspects, the probes are provided on a platform, such as a microarray, to form a panel. In some aspects, the panel further contains probes for other histones or proteins useful for disease detection or prognosis, as known in the art.

### Actions Based on Identifications

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc.

For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising making one or more further identifications based on one or more other identifications. For example, particular treatments, monitoring, follow-ups, advice, etc. can be identified based on the other identification. For example, identification of a subject as having a disease or condition with a high level of a particular component or characteristic can be further identified as a subject that could or should be treated with a therapy based on or directed to the high level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods comprising treating, monitoring, following-up with, advising, etc. a subject identified in any of the disclosed methods. Also disclosed are methods comprising treating, monitoring, following-up with, advising, etc. a subject for which a record of an identification from any of the disclosed methods has been made. For example, particular treatments, monitoring, follow-ups, advice, etc. can be used based on an identification and/or based on a record of an identification. For example, a subject identified as having a disease or condition with a high level of a particular component or characteristic (and/or a subject for which a record has been made of such an identification) can be treated with a therapy based on or directed to the high level component or characteristic. Such treatments, monitoring, follow-ups, advice, etc. can be based, for example, directly on identifications, a record of such identifications, or a combination. Such treatments, monitoring, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

The disclosed measurements, detections, comparisons, analyses, assays, screenings, etc. can be used in other ways and for other purposes than those disclosed. Thus, the disclosed measurements, detections, comparisons, analyses, assays, screenings, etc. do not encompass all uses of such measurements, detections, comparisons, analyses, assays, screenings, etc.

### Biomedical Research and Clinical Testing

Histone tyrosine phosphorylation antibodies raised against H2B-Tyr37, H4-Tyr88, H4-Tyr51 and/or H3-Tyr99 can be highly useful in biomedical research which involves sensitive techniques such as immunoassays (e.g., ELISA, immunoblotting, immunoprecipitation, immunohistochemistry), Chip-on-CHIP and ChIP-sequencing.

For example, immunoblotting can be used in research and clinical setting to determine which biological samples contain the disclose phosphorylations that may correlate with disease occurrence, development or progression. Antibodies can also be used in immunoprecipitation experiments to identify interacting proteins or proteins that partner with them to regulate specific biological processes, such as proteins required for cell cycle.

Chromatin Immunoprecipitation followed by hybridization (Chip-on-CHIP) and Chip-sequencing can be used to determine the localization of this modification at specific genomic locations and to determine which genes are targeted and turned on and off in a variety of diseases and disorders such as cancer, diabetes, obesity and diet related disorders.

Histone H2B-Tyr37, H4-Tyr88, H4-Tyr51 and H3-Tyr99 antibodies can be used for analysis of cellular response to growth and proliferation signals of normal and cancer cell types, based on the phosphorylation patterns. The examples are: (a) Response to insulin and insulin like growth factors in cancer, obesity related disorders and in diabetes; and (b) Response to platelet derived growth factors in bone marrow transplants. Blood and tissue biopsies obtained from known cancer, diabetes or obese patients can be screened with the antibodies to detect differences in expression profiles. Significant alterations can be correlated with disease progression.

H2B-Tyr37, H4-Tyr88, H4-Tyr51 and H3-Tyr99 antibodies can be used to analyze patient samples after radiotherapy and chemotherapy to determine the effect of the treatment on gene expression and cellular proliferations.

Tagged, for example with a detectable label such as a fluorescent tag, H2B-Tyr37, H4-Tyr88, H4-Tyr51 and H3-Tyr99 antibodies can be used to isolate protein complexes of interest that may be required for specific biological process such as embryo development or patterning in various organisms, such as yeasts, worms, flies, fishes, frogs, mice, and humans.

H2B-Tyr37, H4-Tyr88, H4-Tyr51 and H3-Tyr99 antibodies can be used in global genomic, metabolomic and proteomics studies to identify candidate genes that are regulated by the histone modification in various organisms, such as yeasts, worms, flies, fishes, frogs, mice, and humans.

Genes associated with phosphorylated H2B-Tyr37, H4-Tyr88, H4-Tyr51 and/or H3-Tyr99 can be identified and assessed. For example, changes in expression of genes associated with phosphorylated H2B-Tyr37, H4-Tyr88, H4-Tyr51 and/or H3-Tyr99 can be used to detect, diagnose, prognose, etc. cancer and other diseases. Examples of genes and genomic sequences associated with phosphorylated H2B-Tyr37, H4-Tyr88, H4-Tyr51 and/or H3-Tyr99 are observed. As described herein, association of phosphorylated H2B-Tyr37, H4-Tyr88, H4-Tyr51 and/or H3-Tyr99 with genes affects expression of these genes and can thus affect disease. Thus, for example, detection of certain expression levels and/or changes in expression levels of genes associated with phosphorylated H2B-Tyr37, H4-Tyr88, H4-Tyr51 and/or H3-Tyr99 can be used in all of the ways and for all of the purposes disclosed herein for detection of associated with phosphorylated H2B-Tyr37, H4-Tyr88, H4-Tyr51 and/or H3-Tyr99.

All of the methods disclosed herein can be used in and with any relevant cells, tissues, organs, organisms, etc. to assess, for example, histone phosphorylation, gene and chromatin associations of phosphorylated histones, and the effect of histone phosphorylation and gene association on gene expression, epigenetic phenotypes, physiology, and disease conditions, progression, etc. The disclosed phosphospecific probes, such as the disclosed antibodies, are especially useful for studying epigenetic effects of histone phosphorylation at a basic level in experimental organisms.

### Immunoassays

In some embodiments, the disclosed phosphospecific probes are antibodies, which are used in an immunoassay to detect a phosphorylated histone. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immuoprecipitation assay (IP), immunobead capture assays, Western blotting, dot blotting, gel-shift assays, ChIP, ChIP-on-CHIP, ChIP-sequencing, flow cytometry, protein arrays, antibody arrays, multiplexed bead arrays, magnetic capture, in vivo imaging, fluorescence resonance energy transfer (FRET), and fluorescence recovery/localization after photobleaching (FRAP/FLAP).

Immunoassays can include methods for detecting or quantifying the amount of a molecule of interest (such as phosphorylated histones) in a sample, which generally involves the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

### Diagnosing, Prognosing, and Treating Disease

Methods for diagnosing a disease in a subject are provided that involve assaying a sample from the subject for phosphorylation of human Histone H2B tyrosine 37 residue, human Histone H4 tyrosine 51 residue, or human Histone H3 tyrosine 99 residue.

In some embodiments, the disclosed epigenetic changes can be reversed by drugs and therefore are good targets for the prevention and treatment of disease. The field of epigenetics is inspiring the discovery of new drugs, and is gaining importance as part of toxicology testing during drug development. Epigenetic therapy, the use of drugs to correct epigenetic defects, is relatively new and rapidly developing area of pharmacology. Epigenetic therapy is a potentially very useful form of therapy because epigenetic defects, when compared to genetic defects, are thought to be more easily reversible with pharmacological intervention. In addition to holding promise as therapeutic agents, epigenetic drugs may also be able to prevent disease.

To assess the effect of histone H2B-Tyr37, H4-Tyr88, H4-Tyr51 and H3-Tyr99 phosphorylations on health and disease, epigenetic variations were have catalogued across the genome or epigenome in different tissues and at various stages of development. Epigenetic changes can be detected in several ways. One method uses chromatin immunoprecipitation, or ChIP. This involves crosslinking DNA with its associated proteins and then shearing the DNA. The fragments that contain H2B-Tyr37, H4-Tyr88, H4-Tyr51 and H3-Tyr99 phosphorylations are extracted by immunoprecipitation with antibodies specific for H2B-Tyr37, H4-Tyr88, H4-Tyr51 and H3-Tyr99 phosphorylations. The immunoprecipitated DNA is purified and labeled with a fluorescent tag. This is then applied to the surface of a DNA microarray containing a set of probes—a procedure commonly referred to as ChIP-on-chip. The purified ChIP-DNA can also be sequenced, called as ChIP-sequencing.

Using ChIP-on-CHIP and ChIP-sequencing approach, about 3500 distinct sites in genome which have histone H2B-Tyr37 phosphorylation were identified. Further, about 500 distinct sites in genome which have histone H4-Tyr51 phosphorylation were identified. The genes proximal to these sites (in some cases sites are located within genes) are likely to be modulated by H2B-Tyr37 or H4-Tyr51 phosphorylations, respectively.

Availability of highly specific antibodies and the knowledge of the sites at which histone are modified would allow for the development of inhibitors (drugs) that suppresses H2B-Tyr37, H4-Tyr88, H4-Tyr51 and H3-Tyr99 phosphorylations in epigenome.

The kinase WEE1 is primarily responsible for histone phosphorylations at H2B-Tyr37. The tyrosine kinases Ack1 and EGFR are primarily responsible for histone phosphorylations at H4-Tyr88, H4-Tyr51 and H3-Tyr99. The specific inhibitors that suppress ability of WEE1 and Ack1 to phosphorylate H2B at Tyr37, H4 at Tyr88 and H3 at Tyr99 would therefore be therapeutically useful, especially in those patients where phosphorylation of regulatory regions of genes is an established epigenetic change known to occur. This ‘personalized therapy’ would bring in high benefits and reduce tumor related deaths.

The disclosed method system can further involve the use of a computer system to compare levels of the one or more of the disclosed biomarkers to control values. For example, the computer system can use an algorithm to compare levels of two or more biomarkers and provide a score representing the risk of disease onset based on detected differences. Therefore, also provided is an apparatus for use in diagnosing, prognosing, or selecting a therapy in a subject that includes an input means for entering phosphorylated histone level values from a sample of the subject, a processor means for comparing the values to control values, an algorithm for giving weight to specified parameters, and an output means for giving a score representing the risk of disease onset.

### Cancer

Most cancers are a mixture of genetic and epigenetic changes. Although it is now well recognized that in most of cancers the epigenetics changes play a crucial role, the identities of precise histone phosphorylation events were not known, and the tools, e.g., phosphorylation-specific antibodies, were not available.

Therefore, the disclosed methods can be used to diagnose, prognose, and/or treat cancer. In some embodiments, the cancer of the disclosed methods can be any cell in a subject undergoing unregulated growth. In preferred embodiments, the cancer is any cancer cell capable of metastasis. For example, the cancer can be a sarcoma, lymphoma, leukemia, carcinoma, blastoma, or germ cell tumor. A representative but non-limiting list of cancers that the disclosed compositions can be used to detect include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

Disclosed are methods of treating cancer in a subject that involves first performing a method as disclosed herein, which in some embodiments, comprises, or alternatively consists essentially of, or yet further consists of, contacting a sample isolated from the subject with one of more of the disclosed phosphospecific probes or antibodies.

Also disclosed are methods of treating a subject in need thereof and selected for the therapy comprising, or alternatively consisting essentially of, or yet further consisting of, administering an ACK1 inhibitor to a subject in which phosphorylated H3-Tyr99, H4-Tyr88 and/or H4-Tyr51 was detected.

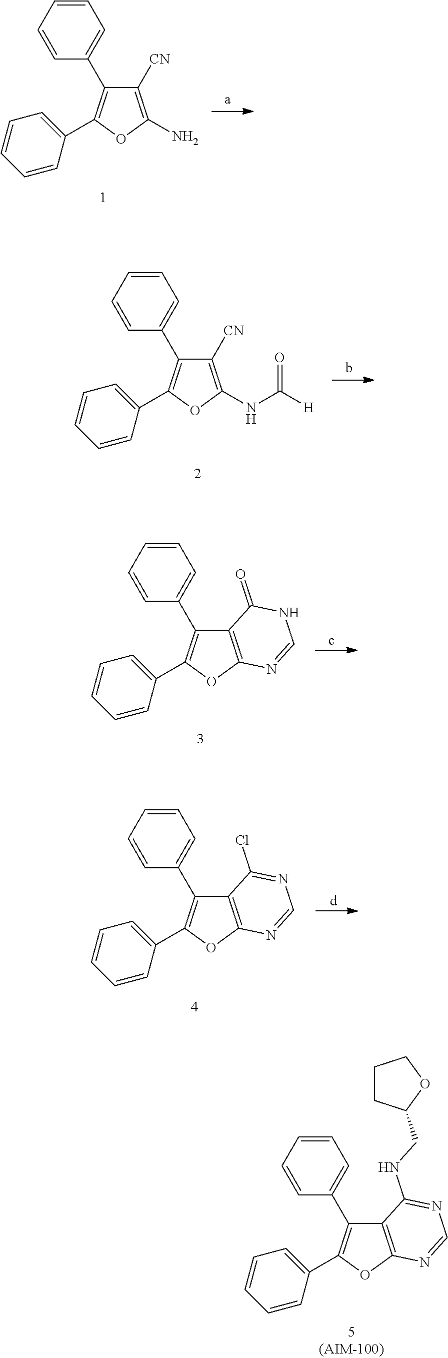

Inhibitors for WEE1 and ACK1 are known in the art; so are methods of preparing them. For instance, to prepare AIM-100, an Ack1 inhibitor, synthesis can start from commercially available compound 1 (shown above). (a) Ac2O, HCOOH, 60° C., 6 hr, followed by slow addition of 1 at 0° C. then rt 12 hr, 90%; (b) AcOH, microwave heating at 200° C., 60 min, 75%; (c) POCl3, 55° C., 2 hr, under argon, 100%; (d) (S)-(+)-Tetrahydrofurfurylamine, EtOH, reflux, 5 hr, 87%.

### EXAMPLES

### Example 1

### Generation of Antibodies

Generation and Affinity Purification of pTyr37-H2B Antibody

Two H2B peptides coupled to immunogenic carrier proteins were synthesized as shown below and pTyr37-H2B antibodies were custom synthesized by 21^{st} century Biochemicals, MA.

The phosphopeptide:

| | |
|---|---|
| Ac-KRSRKES[pY]SVYVYKVL-Ahx-C-amide. | (SEQ ID NO: 9) |
| The non-phospho peptide: | |
| Ac-KRSRKESYSVYVYKVL-Ahx-C-amide. | (SEQ ID NO: 10) |

In brief, two rabbits were immunized twice with the phosphopeptide, several weeks apart, and enzyme-linked immunosorbent assay was performed to determine the relative titer of sera against phosphorylated and nonphosphorylated peptides. The titer against phosphorylated peptides (˜1:40,000) was much greater than nonphosphorylated peptide (1:2000). The sera were affinity purified. Two antigen-affinity columns were used to purify the phospho-specific antibodies. The first column was the non-phosphopeptide affinity column. Antibodies recognizing the unphosphorylated residues of the peptide bound to the column and were eluted as pan-specific antibodies. The flow-through fraction was collected and then applied to the second column, the phosphopeptide column. Antibodies recognizing the phospho-residue bound to the column and were eluted as phospho-specific antibodies. The antibodies were extensively validated for its specificity by immunoblottings.

Antibodies against H3 Tyr99 and H4 Tyr51 and Tyr88 were produced using the same techniques (but substituting appropriate H3 and H4 peptides, respectively, for immunization of rabbits).

### Native Chromatin Immunoprecipitation (ChIP)

As a first step, extensive standardization of pTyr37-H2B antibodies were performed for its usage in ChIP. CHIP was performed using the Active Motif kit as per manufacturer's instructions. For ChIP synchronized MEFs were harvested at 0 and 6.30 hour post thymidine release (5×10⁷ cells). Cells pellets were lysed in RLB buffer (Mahajan, N. P. et al. (2007) Proc. Nat. Acad. Sci. U.S.A. 104:8438-8443; Mahajan, K. et al. (2010) Prostate 70:1274-1285) on ice for 10 minutes and sonicated for 25 seconds to shear DNA to an average length of 300-500 bp. The soluble chromatin was incubated overnight at 4° C. with pTyr37-H2B antibody (also pTyr88-H4 antibody and pTyr99-H3 antibody). 20 μl of protein-A agarose was added and the beads were washed sequentially and DNA was eluted. Genomic DNA (Input) was prepared by treating aliquots of chromatin with RNase, proteinase-K followed by ethanol precipitation. Pellets were resuspended and the resulting DNA was quantified on a NanoDrop spectrophotometer. Extrapolation to the original chromatin volume allowed quantitation of the total chromatin yield. An aliquot of chromatin (20-30 μg) was pre-cleared with protein-A agarose beads (Invitrogen). Genomic DNA regions of interest were isolated using pTyr37-H2B antibody (also pTyr88-H4 antibody and pTyr99-H3 antibody). After incubation at 4° C. overnight, protein-A agarose beads were used to isolate the immune complexes. Complexes were washed, eluted from the beads with SDS buffer, and subjected to RNase and proteinase-K treatment and ChIP DNA was purified by phenol-chloroform extraction and ethanol precipitation.

### ChIP-on-Chip

ChIP and Input DNAs were amplified by whole-genome amplification (WGA) using the GenomePlex WGA Kit (Sigma). The resulting amplified DNAs were purified, quantified, and tested by QPCR at the same specific genomic regions as the original ChIP DNA to assess quality of the amplification reactions. Amplified DNAs were fragmented and labeled using the DNA Terminal Labeling Kit from Affymetrix, and then hybridized to Affymetrix GeneChip Tiling or Promoter arrays at 45° C. overnight. Arrays were washed and scanned, and the resulting CEL files were analyzed using Affymetrix TAS software. Thresholds were selected, and the resulting BED files were analyzed (using Genpathway proprietary software) that provides comprehensive information on genomic annotation, peak metrics and sample comparisons for all peaks (intervals).

### Native ChIP-Sequencing and Analysis

The native ChIP was performed as described above. The 0 and 6.30 hrs post thymidine release chromatin immunoprecipitated DNAs were subjected to sequencing. Sequencing yield was very good with almost 40 million reads in each sample, of which 27.7 and 23.6 million for samples 6.30 hr and 0 hr, respectively, mapped uniquely to the mouse mm9 genome.

a. Sequence Analysis:

The 36-nt sequence reads (“tags”) identified by the Sequencing Service (using Illumina's Genome Analyzer 2) are mapped to the genome using the ELAND algorithm. Alignment information for each tag is stored in the output file *_export.txt. Only tags that map uniquely, have no more than 2 mismatches, and that pass quality control filtering are used in the subsequent analysis.

b. Determination of Fragment Density:

Since the 5″-ends of the sequence tags represent the end of ChIP/IP-fragments, the tags were extended in silico (using Active Motif software) at their 3′-ends to a length of 110-200 bp, depending on the average fragment length in the size selected library. To identify the density of fragments (extended tags) along the genome, the genome was divided into 32-nt bins and the number of fragments in each bin was determined. This information was stored in a BAR (Binary Analysis Results) file that can be viewed in a browser such as Affymetrix′ Integrated Genome Browser (IGB).

c. Interval Analysis (“Peak Finding”):

An Interval is a discrete genomic region, defined by the chromosome number and a start and end coordinate. Intervals represent the locations of fragment density peaks. For each BAR file, Intervals are calculated and compiled into BED files (Browser Extensible Data). A typical threshold setting is in the range of 10-20, but may be adjusted depending on the number of tags sequenced or based on information on positive and negative test sites, independent estimates for the false discovery rate (FDR), and/or the intent to generate a stringent or relaxed analysis. The applied threshold can be found in the Assay Results Report. For an Interval to be called, it must contain 3 consecutive bins with fragment densities greater than the threshold.

d. Alternative and/or Optional Analysis Steps:

1. Tag Normalization: When samples had uneven tag counts, the tag numbers of all the samples were truncated to the number of tags present in the smallest sample.

2. False Peak Filtering: Input or IgG control sample (which represent false peaks) were used to remove corresponding Intervals in ChIP samples, or to mark them as likely false positives.

3. MACS: This alternative, model-based peak finding algorithm (Zhang, Y. et al. (2008) Genome Biol. 9:R137) was used if an Input or IgG control sample was available.

e. Active Region Analysis:

To compare peak metrics between 2 or more samples, overlapping Intervals were grouped into “Active Regions”, which were defined by the start coordinate of the most upstream Interval and the end coordinate of the most downstream Interval (=union of overlapping Intervals). In locations where only one sample had an Interval, this Interval defined the Active Region. Active Regions were useful to consider because the locations and lengths of Intervals were rarely exactly the same when comparing different samples.

f. Annotations:

After defining the Intervals and Active Regions, their exact locations along with their proximities to gene annotations and other genomic features were determined and presented in Excel spreadsheets. In addition, average and peak fragment densities within Intervals and Active Regions were compiled.

### Pull Down and Filter Binding Assay

Two human histone H2B peptides spanning amino acids 25-49 were synthesized with Tyr37 at middle of the peptide. The sequences are as follows:

| H2B(25-49): | |
|---|---|
| DGKKRKRSRKESYSVYVYKVLKQVH | (SEQ ID NO: 11) |
| pY37-H2B(25-49): | |
| DGKKRKRSRKESpYSVYVYKVLKQVH | (SEQ ID NO: 12) |

Both the peptides were biotinylated at C-terminus and immobilized on streptavidin-sepharose beads. The beads were incubated with HEK293 cell lysates made in TGN buffer containing 50 mmol/L Tris (pH 7.5), 50 mmol/L Glycine, 150 mmol/L NaCl, 1% Triton X-100, 10% glycerol, phosphatase inhibitors (10 mmol/L NaF, 1 mmol/L Na₂VO₄), and protease inhibitor mix (Roche). The beads were extensively washed with TGN buffer and bound NPAT was resolved by SDS-PAGE followed by immunoblotting with NPAT antibodies. Equal loading of peptide was determined by Coomassie blue staining.

NPAT binding to unphosphorylated H2B was confirmed by filter binding assay. Two concentrations of H2B(25-49) or pY37-H2B(25-49) peptides were spotted on nitrocellulose membrane which was incubated with HEK293 cell lysates prepared in TGN buffer. Blot was washed extensively followed by immunoblotting with NPAT antibodies.

### Example 2

### Histone H2B Phosphorylation at Tyrosine 37 by WEE1 Kinase

The precise orchestration of epigenetic signaling networks ensures timely gene expression profiles that are critical to safeguard against catastrophic cellular events. Components of these epigenetic signaling pathways include writers, readers and erasers, each of which plays a critical role in regulated gene expression. Importantly, deregulation of epigenetic mechanisms is linked to cancer, hereditary and metabolic diseases (Probst, A. V. et al. (2009) Nat. Rev. Mol. Cell. Biol., 10(3):192-206; Schwartzentruber, J. et al. (2012) Nature 482(7384):226-231; Wu, G. et al. (2012) Nature Genetics 44(3):251-253; Sturm, D. et al. (2012) Cancer Cell 22(4):425-437; Brower, V. (2011) Nature 471(7339):S12-S13; Burgess, R. J. et al. (2013) Nat. Struct. Mol. Biol. 20(1):14-22). Applicant recently discovered the existence of a novel epigenetic signaling network wherein WEE1 kinase directly phosphorylates the histone H2B (pY37-H2B) to cause repression of global histone synthesis, precisely in the late S phase of the cell cycle (Mahajan, K. et al. (2012) Nat. Struct. Mol. Biol. 19(9):930-937). Mechanistically, it was observed that pY37-H2B epigenetic marks recruit the HIRA transcriptional repressor, exclude the transcriptional co-activator, NPAT, to temporally suppress mRNA synthesis, providing a key evidence of how cells use this epigenetic signaling pathway to precisely coordinate duplication of DNA and histones during each cell cycle (Mahajan, K. et al. (2012) Nat. Struct. Mol. Biol. 19(9):930-937; Mahajan, K. et al. (2013) Trends in Genetics: TIG 29(7):394-402). Consonantly, it was observed that a WEE1-specific small molecule inhibitor, MK-1775 (now AZD-1775) not only inhibited WEE1 epigenetic activity but also robustly reversed histone transcriptional suppression, in both yeast and mammalian cells, indicating that WEE1/pY37-H2B epigenetic signaling has an universal applicability (Mahajan, K. et al. (2012) Nat. Struct. Mol. Biol. 19(9):930-937; Mahajan, K. et al. (2013) Trends in Genetics: TIG 29(7):394-402). Based on these data and without being bound by theory, applicant suggests that WEE1/pY37-H2B epigenetic signaling plays a critical role in chromatin replication by silencing histone transcription that can be reversed by WEE1 inhibitor, e.g., MK-1775 (AZD-1775). Applicant also established the novel epigenetic reader function of HIRA and its role in suppression of global histone output. Further, it was demonstrate that the reversal of pY37-H2B epigenetic marks by MK-1775 could overcome ‘loss of heterochromatinization’ caused by abridged histone synthesis in WEE1 overexpressing cancer cells.

Serendipitously, applicant discovered that the pY37-H2B marks are instantaneously erased after IR-induced DNA damage and are restored after about 2 hours when majority of the DSBs are repaired. The removal of these marks was found to be dependent on the activity of Ataxia telangiectasia mutated (ATM) kinase, a master regulator of the DNA damage signaling pathway. Thus, these data indicate that another regulatory layer is involved in the deposition of pY37-H2B epigenetic marks. Applicant investigated how these marks are erased in a timely manner by EYA and CDC14 tyrosine phosphatases.

Interestingly, in light of Applicant's recent discovery, the evolutionarily conserved WEE1 epigenetic function acquires a new dimension—WEE1 is aberrantly expressed in highly aggressive tumors such as glioblastoma multiforme (GBMs), malignant melanomas and triple-negative and luminal breast cancers (Mir, S. E. et al. (2010) Cancer Cell 18(3):244-257; Wuchty, S. et al. (2011) PloS One 6(2):e14681; Magnussen, G. I. et al. (2012) PloS One 7(6):e38254; Aarts, M. et al. (2012) Cancer Discov. 2(6):524-539; Iorns, E. et al. (2009) PloS One 4(4):e5120). To decipher its puzzling role in malignancy, Applicant mined the ChIP-sequencing data which revealed that in addition to HIST1, pY37-H2B marks are also deposited at a tumor suppressor gene—the isocitrate dehydrogenase 2 (IDH2). This gene encodes a key enzyme in the pathway catalyzing the conversion of methyl group at the 5′ position of cytosine (5-mC) to 5-hydroxymethylcytosine (5-hmC) (Xu, W. et al. (2011) Cancer Cell 19(1):17-30)—a metabolite significantly reduced in malignant cancers (Haffner, M. C. et al. (2011) Oncotarget 2(8):627-37; Jin, S. G. et al. (2011) Cancer Res. 71(24):7360-7365; On, B. A. et al. (2012) PLoS One 7(7):e41036; Lian, C. G. et al. (2012) Cell 150(6):1135-1146). While IDH2 mRNA expression is down regulated in brain and skin cancers, the mechanistic basis of its transcriptional suppression is not known. Therefore, Applicant profiled 27 primary GBM biopsies and 6 normal brain samples and observed that a subset of the GBMs (about 26%) exhibited elevated WEE1 mRNA levels coupled with a striking downregulation of IDH2 mRNA transcription.

Therefore, Applicant demonstrated that by overexpressing WEE1, cancer cells suppress IDH2 gene expression—revealing a novel epigenetic pathway wherein histone H2B Tyr37-phosphorylation regulates DNA methylation, promoting GBM and melanoma malignancy. Overall, pY37-H2B modification was studied at two important genetic loci (HIST1 and IDH2) and assessed its role in histone transcription and gene regulation.

### Epigenetic Mechanisms Underlying IDH2 Transcriptional Down Regulation in Melanoma

Melanoma is a malignant tumor of the melanocytes which causes the majority (75%) of deaths related to skin cancer. According to American Cancer Society, about 76,690 new melanomas will be diagnosed in United States in 2013 and about 9,480 people are expected to die of melanoma (Siegel, R. et al. (2013) CA Cancer J. Clin. 63(1):11-30). About 50-60% of melanomas contain a mutation (V600E or rarely V600K/R/M) in the B-Raf gene (Davies, H. et al. (2002) Nature 417(6892):949-954). Clinical trials suggested that BRAF inhibitors including Vemurafenib could lead to substantial tumor regression in a majority of patients if their tumor contains the BRAF mutation (Chapman, P. B. et al. (2011) N. Engl. J. Med. 364(26):2507-2516). However, the molecular basis of pathogenesis in remaining 40-50% of melanoma patients is not fully understood, which has made research into understanding the biology and the identification of therapeutic targets an area of intense research interest.

Isocitrate dehydrogenases, IDH1 and IDH2, catalyze the oxidative decarboxylation of isocitrate to α-ketoglutarate (α-KG), a crucial regulator of cell metabolism (Xu, W. et al. (2011) Cancer Cell 19(1):17-30; Reitman, Z. J. et al. (2010) J. Natl. Cancer Inst. 102(13):932-941). About 60 dioxygenases expressed in mammalian cells utilize α-KG as an essential cofactor in the oxidation reaction, including the recently discovered TET family of 5-methylcytosine (5mC) hydroxylases that convert 5-mC to 5-hmC (Tahiliani, M. et al. (2009) Science 324(5929):930-935). In recent years, the loss of 5-hmC has been identified to be a recurrent epigenetic hallmark in melanomas and GBMs (Orr, B. A. et al. (2012) PLoS One 7(7):e41036; Lian, C. G. et al. (2012) Cell 150(6):1135-1146; Krell, D. et al. (2011) PloS One 6(5):e19868). A clue to IDH2 down regulation in melanoma emerged from the ChIP-Sequencing experiments—it revealed that WEE1 deposits pY37-H2B marks within the IDH2 gene. It indicated that WEE1 is not only crucial in regulating G2/M transition, its well-established role, but possesses an additional ‘arrow in quiver’, a histone modification activity which regulates ‘other’ epigenetic modifications, such as DNA methylation.

Without being bound by theory, proposed are mechanisms by which WEE1 epigenetically suppresses the expression of IDH2 gene and prevents the formation of 5-hmC, which in turn alters the epigenetic landscape, thereby promoting cancer cell proliferation.

Metastatic Melanomas Display Significant Decrease in IDH2 mRNA Expression

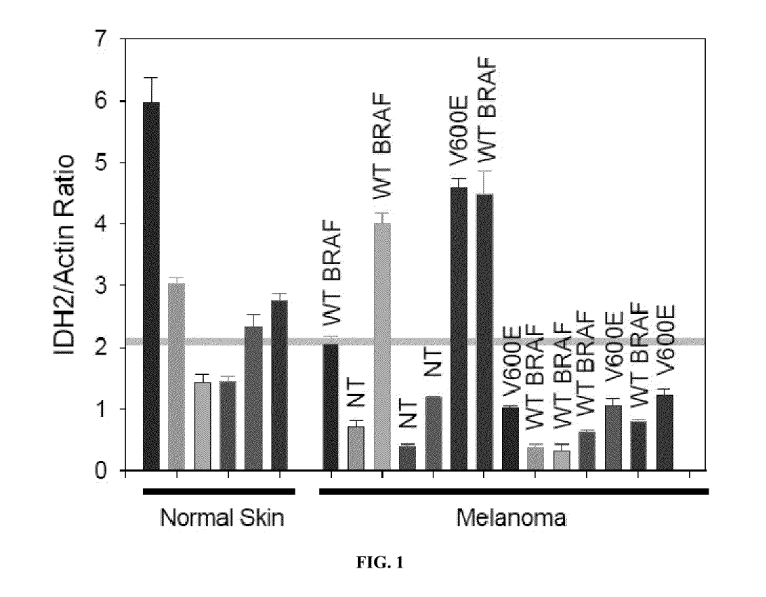

Not only is WEE1 overexpressed in melanomas, MK-1775, a potent WEE1-specific inhibitor selectively induces apoptosis in WEE1 expressing cancer cell lines (Hirai, H. et al. (2009) Mol. Cancer Ther. 8(11):2992-3000), suggesting that some of the melanomas may be addicted to the WEE1 signaling pathway for survival. Based on the finding that WEE1 epigenetically regulates the expression of chromatin modifying gene IDH2, Applicant envisaged that alterations in WEE1 regulatory control could significantly disrupt the epigenetic landscape of melanomas to promote malignancy. To interrogate this hypothesis further, applicant obtained 14 primary melanomas and 6 normal skin samples (total 18 samples) under SRC/IRB approved protocol, MCC#15375, IRB Study #106509. All the tumors were microdissected and pathologist validated each of the microdissected tissue samples prior to its usage. As a first step, total RNA was prepared followed by qRT-PCR using IDH2 and actin specific primers. Although, there was some inter individual variability, a significant decrease in IDH2 mRNA levels was apparent in melanomas as compared to normal skin samples (FIG. 1).

Statistical Analysis:

Relative expression of IDH2 mRNAs was determined based on its ratio with actin. The log-transformation was taken so that the data were normally distributed. The differences in relative IDH2 mRNA levels between melanoma and normal human skin were statistically significant (p=0.048). The p-values are two-sided and computed by the two-sample t-test. To identify patients that exhibit significant downregulation of IDH2, the threshold for each variable was selected to maximize the sensitivity (ratio of positives to melanoma) when the specificity (ratio of negatives to normal samples) is set to 1. These indicated that the melanoma patients whose IDH2/Actin ratios are <2.1, are likely to have active/elevated WEE1/pY37-H2B signaling (FIG. 1). There were 10 such patients (patient #1, 9, 10, 11, 13) that fit in this category, indicating that about 70% (5 of 7) of the melanoma patients exhibit elevated WEE1/pY37-H2B signaling (90% CI: 0.128-0.432).

Translational Significance—BRAF Mutation Negative (or BRAF WT) Melanoma Exhibit H2B pTyr37-H2B/WEE1/IDH2 signaling

BRAF is an oncogene that encodes a serine/threonine-protein kinase B-Raf. Somatic mutations in BRAF gene have been found in ˜50% of all malignant melanomas (COSMIC; Davies et al. (2002); Maldonado et al. (2003)). These patients often respond to BRAF inhibitor therapy which consists of vemurafenib or dabrafenib, both of which are approved by FDA for treatment of late-stage melanoma.

However, the remaining 50 percent of melanoma patients without BRAF mutations (or BRAF WT) do not benefit from BRAF inhibitors and remain a challenge to treat. Since there has not been any effective treatment for melanoma patients with wildtype (WT) BRAF, applicants have explored these melanomas for further study and observed that 5 out of 7 melanoma patients with WT BRAF, including one with NRas mutation, exhibited suppression of IDH2. This data is consistent with the human cell line data that was observed (shown below). Significantly, Applicant observed that WEE1 inhibitor, MK-1775 reversed epigenetic marks increasing IDH2 transcript levels in melanoma cancer cells.

Overall these data show that selecting melanoma patients that are positive for WEE1 epigenetic signaling could be a ‘companion diagnostic’ strategy for MK-1775 or AZD-1775 treatment for the melanoma patients that lacks BRAF mutations.

Statistical Analysis:

Relative expression of WEE1 and IDH2 mRNAs was determined based on its ratio with actin. The log-transformation was taken so that the data were normally distributed. The descriptive statistics of the raw and the transformed data is shown in Table 1. The differences in relative WEE1 and IDH2 mRNA levels between GBMs and normal human brains were statistically significant (p<0.001 and p=0.028, respectively). The p-values are two-sided and computed by the two-sample t-test. The relative WEE1 expression is predictive of progression to GBM and the odds of progression to GBM increases with increase in relative WEE1 expression (OR=8.7; 95% CI: 1.7-43.4; Area under ROC curve=0.895).

TABLE 1
Descriptive Statistics- IDH2 and WEE1 expression profiles

| Variable | Sample | N | Mean | SD | Med. | Min. | Max |
|---|---|---|---|---|---|---|---|
| IDH2/Actin | GBMs | 27 | 2.4 | 2.39 | 1.61 | 0.37 | 9.3 |
| | Normals | 6 | 5.77 | 4.73 | 4.99 | 1.4 | 13.99 |
| WEE1/Actin | GBMs | 27 | 1.3 | 1.1 | 0.79 | 0.17 | 3.96 |
| | Normals | 6 | 0.28 | 0.27 | 0.17 | 0.06 | 0.76 |

Legend: Med. = median; Min. = minimum; Max = maximum.

### Example 3

### Role of ACK1 Mediated Histone H3 Tyrosine99-Phosphorylation in Regulation of SLC6A4 (Serotonin Transporter or SERT)

### ACK1 is an Epigenetic Kinase

Applicant reported in International Application No. PCT/US2013/020395, filed Jan. 4, 2013, Applicant observed that ACK1 phoshorylates histone H3 at Tyrosine 99 residue). Since the functional role of Y99-phosphorylated H3 (pY99-H3) is unknown, phospho-antibodies were raised against pY99-H3 which has been extensively validated.

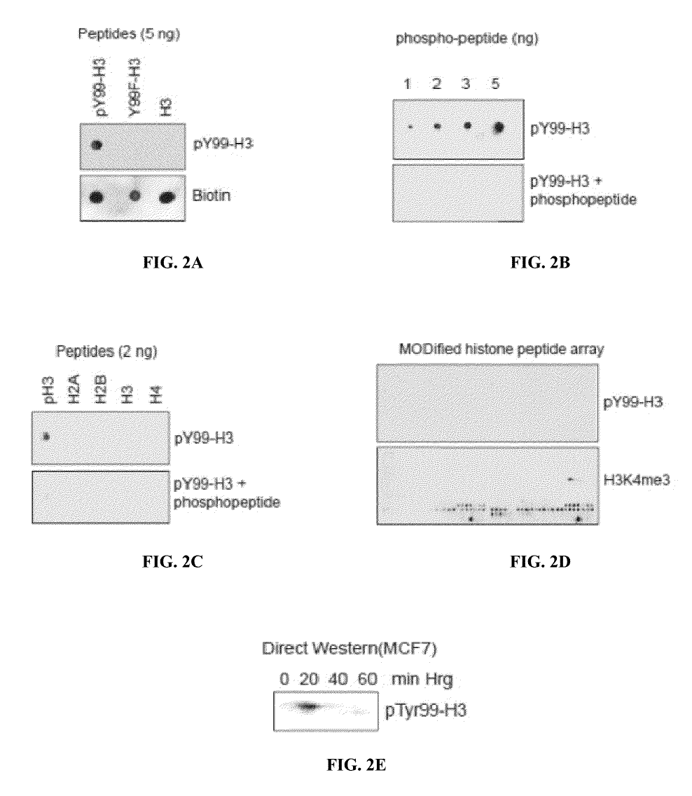

Applicant compared recognition of the peptides by pY99-H3 antibodies and observed that only the H3 phosphopeptide that was phosphorylated at Y99 was recognized whereas the non-phosphorylated H3 peptide or H3 harboring a Tyr99 to Phe (Y37F) substitution was not reactive (FIG. 2A). Further, competition of pY99-H3 antibodies with phosphopeptide resulted in almost complete loss of recognition of Y99-H3 phosphopeptides (FIGS. 2B and 2C, lower panels). Moreover, pY99-H3 antibodies were screened for cross-reactivity against 59 distinct acetylation, methylation, phosphorylation, and citrullination modifications on core histones using Histone Peptide Arrays. The pY99-H3 antibody did not cross-react with any of these PTMs, however when the same blots were hybridized with H3K4me3 antibodies, it revealed the expected pattern of hybridization (FIG. 2D). Immunoblotting with whole cell extract was also performed which revealed a band of ˜17 kDa (FIG. 2E). Collectively, these data indicate that the antibodies are selective for Y37-phosphorylation on histone H3.

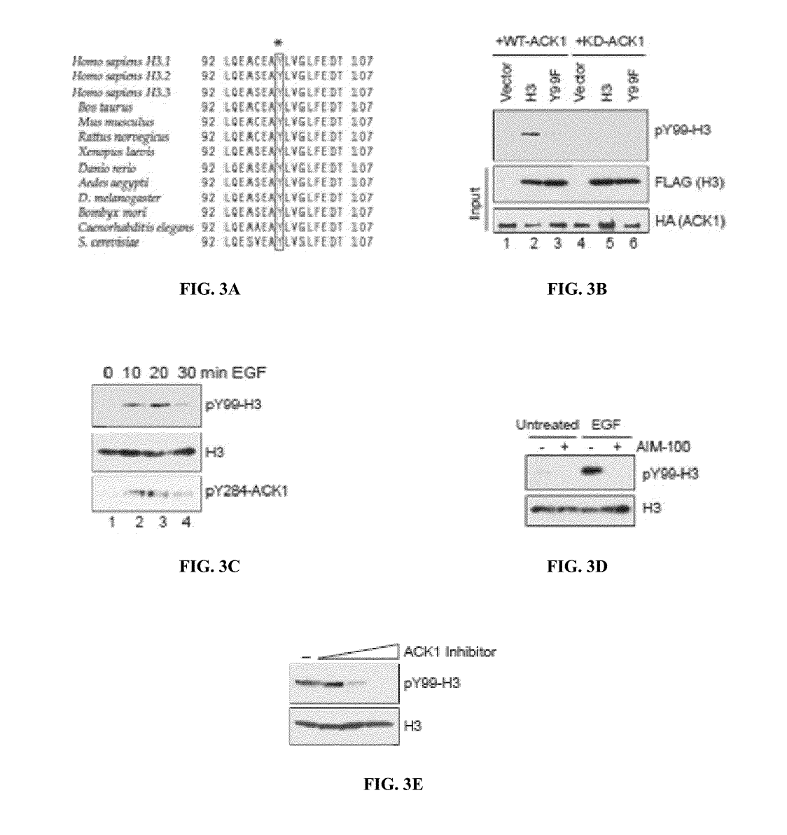

The Tyrosine99 or Y99 site in H3 protein is evolutionarily conserved (FIG. 3A). To examine whether H3 is ACK1 kinase substrate, HEK293 cells were co-transfected with HA-tagged ACK1 and FLAG-tagged H3 or FLAG-tagged Y99F mutant H3 expressing constructs. ACK1 specifically phosphorylated H3 protein, but not the Y99F mutant protein (FIG. 3B). To determine endogenous H3 Tyr99-phopshorylation, HEK293 cells were treated with EGF ligand followed by immunoblotting with pY99-H3 antibodies. EGF ligand mediated ACK1 phosphorylation resulted in significant upregulation in endogenous H3 Tyr99-phosphorylation (FIG. 3C).

To examine whether ACK1 kinase activity is needed for H3 Tyr99-phosphorylation, cells were treated with ACK1 inhibitor, AIM-100, overnight Inhibition of ACK1 by AIM-100 resulted in complete loss of histone H3 Tyr99-phosphorylation (FIG. 3D). To further examine whether ACK1 kinase activity is needed for H3 Tyr99-phosphorylation, cells were treated with increasing concentrations of ACK1 inhibitor, AIM-100. A 0.5 μM concentration of ACK1 inhibitor treatment resulted in the complete loss of pY99-H3 (FIG. 3E).

### Identification of ACK1 Signaling Partners in Brain

ACK1, also known as TNK2, is a non-receptor tyrosine kinase that is highly expressed in the brain (Manser, E. et al. (1995) J. Biol. Chem. 270(42):25070-25078; La Torre, A. et al. (2006) Gene Expr. Patterns 6(8):886-892; Urena, J. M., et al. (2005) J. Comp. Neurol. 490(2):119-132). ACK1 is a molecular constituent of neurotrophin signaling cascades in neurons. ACK1 overexpression induces neuritic outgrowth and promotes branching in neurotrophin-treated neuronal cells, whereas the expression of Ack1 dominant negatives or short-hairpin RNAs counteract neurotrophin-stimulated differentiation, suggesting that Ack1 acts as a novel regulator of neurotrophin-mediated events in primary neurons and in PC 12 cells (La Torre, A. et al. (2013) Cell Death Dis. 4:e602). ACK1 is highly expressed in the central nervous system (CNS) during adulthood and in developing neurons. In neuronal cultures and in vivo, ACK1 is localized in developing dendrites and axons, including growth cones, presynaptic terminals, and dendritic spines (La Torre, A. et al. (2006) Gene Expr. Patterns 6(8):886-892; Urena, J. M., et al. (2005) J. Comp. Neurol. 490(2):119-132).

To comprehensively understand the precise mechanistic details of ACK1 signaling in brain, an ACK1 knockout (KO) mice were generated as described below. The Ack1/TNK2 gene is comprised of 16 exons. Using BAC recombineering method, Applicant cloned 14.854 kb of TNK2 gene into the pBSDTRIXh vector. The ATG codon is located in exon 2. This region is followed by the frt-flanked PGK promoter driven SV40-neo gene, located in intron 2. The 5′ loxP site is located in intron 2 and the 3′ loxP site is inserted in intron 3. Cre-mediated recombination between these two sites results in deletion of part of intron 2 and all of exon 3 to generate a stop codon TGG due to splicing of exon 2 with exon 4, causing premature termination. The targeting construct was electroporated into C57BL/6 (black) embryonic stem (ES) cells. Cells containing the correctly targeted allele were identified by PCR using a forward primer in the genome, outside the region of targeting and reverse primer in the neomycin gene. Neo and 5′ loxP sites were introduced into the second intron along with a new BamHI site just after 3′ loxP in intron 3. As a result digestion of genomic DNA from G418 resistant clones resulted in the appearance of a 9.8 kb and a 13.5 kb bands corresponding to the wildtype and Neo inserted alleles respectively. 23% of the clones were tested positive by PCR and the clones were reconfirmed by southern blotting (data not shown). To conditionally inactivate the Ack1/Tnk2 gene in mice, two embryonic stem cells (ES) clones (B04 and C04) each containing a single targeted TNK2/Ack1 allele were microinjected into albino C57BL/6 (B6) blastocysts. Four mice with 40-75% of chimera were observed. Chimeric males were then mated with B6 albino females to screen for germ line transmission. The floxed pups were expected to be black. Two black pups, likely to be floxed for Ack1 or Ack1^{flx/wt} were obtained. Ack1^{flx/wt} mice were bred with EIIa-Cre mice to determine whether loss of Ack1 leads to embryonic lethality. The adenovirus EIIa promoter directs expression of Cre recombinase in preimplantation mouse embryos and in nearly all tissues. Ack1 heterozygous mice which were subsequently interbred to obtain homozygous knockout mice were obtained. Although not embryonic lethal, early data obtained from breeding Ack1 heterozygotes reveal that Ack1 homozygous KO pups were born with slightly lower than expected mendelian frequencies (17% versus expected 25%) suggesting loss of Ack1 may exhibit some degree of embryonic lethality. ACK1 KO and wild type (WT) mice brain RNAs were isolated followed by RNA-sequencing. The data in shown in Table 2, which indicates a set of genes that are significantly down or upregulated in ACK1 KO mice, as compared to WT mice. RNA sequencing data has revealed that many genes, e.g., Gabra6, Slc6a2, Slc6a4, Slc6a5 and Oxt are significantly modulated in KO mice, suggesting that ACK1 is a critical regulator of expression of these mRNA and proteins in brain. Significantly, these genes have been identified to be involved in Autism spectrum disorder (ASD) and epilepsy, in both human and mouse.

Interestingly, recently a large scale exome sequencing identified a mutation in ACK1/TNK2 gene in a Belgian-Italian family in which all three children possessing this mutation presented with infantile-onset epilepsy and cognitive regression (Hitomi, Y. et al. (2013) Ann. Neurol. 74(3):496-501). These data indicates that ACK1 may have a role to play in infantile-onset epilepsy and cognitive regression by regulating genes, e.g., Gabra6, Slc6a2, Slc6a4, Slc6a5 and Oxt.

### SLC6A4, a Serotonin Transporter and its Importance

One of the gene that was significantly downregulated in ACK1 KO mice was SLC6A4. SLC6A4 (solute carrier family 6, neurotransmitter transporter, member 4) is a protein-coding gene also known as serotonin transporter or SERT or 5-Hydroxytryptamine (Serotonin) Transporter or 5-HTT or 5-HTTLPR. This gene encodes an integral membrane protein that transports the neurotransmitter serotonin from synaptic spaces into presynaptic neurons. The encoded protein terminates the action of serotonin and recycles it in a sodium-dependent manner. This protein is a target of psychomotor stimulants, such as amphetamines and cocaine, and is a member of the sodium neurotransmitter symporter family. Mood, emotion, cognition, and motor functions as well as circadian and neuroendocrine rhythms, including food intake, sleep, and reproductive activity, are modulated by the midbrain raphe serotonin (5-HT) system. There is evidence to suggest that SERTs are linked to neuroticism, sexual behavior, alcoholism, clinical depression, hypertension and obsessive-compulsive disorder. The molecular mechanism of antidepressant drug action too involves inhibition of the neuronal serotonin transporter SERT, encoded by the gene SLC6A4.

The serotonin transporter (SERT)1 is responsible for reuptake of serotonin (5-HT) released during neurotransmission. Inhibitors of SERT are clinically effective as antidepressants. Psychostimulants such as cocaine and amphetamine derivatives also interact with SERT, either as inhibitors or alternative substrates.

ACK1 KO Mice Exhibit Significant Downregulation of SLC6A4 mRNA and Protein

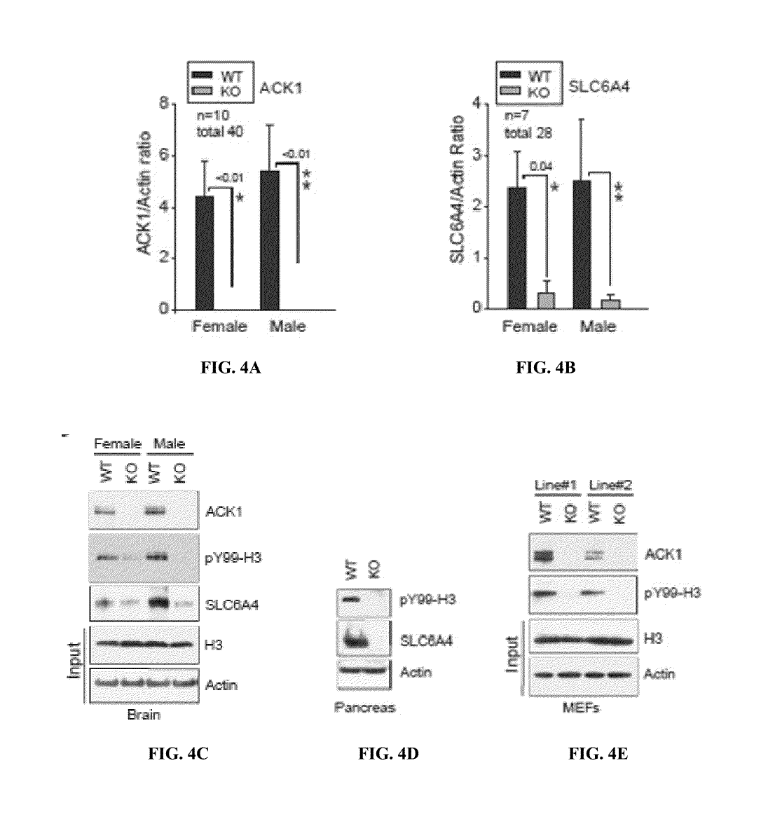

To determine whether ACK1 KO mice have lost ACK1 expression, RNA was isolated from 40 mice brains (10 male KO, 10 Female KO, 10 WT males and 10 WT females) and real time RT-PCR was performed using ACK1 exon 2 specific primers. Complete loss of ACK1 mRNA expression was seen in ACK1 KO mice brains, as compared to WT mice brains (FIG. 4A). To assess whether loss of ACK1 resulted in decreased SLC6A4 mRNA expression, a qRT-PCR was performed using SLC6A4 specific primers. As compared to WT mice brains, the KO mice brains exhibit significant decrease SLC6A4 mRNA levels (FIG. 4B).

To examine whether loss of ACK1 and SLC6A4 mRNA in ACK1 KO mice is also reflected in loss of corresponding proteins, brain lysates of WT and KO mice were immunoblotted. KO mice brains exhibited significant decrease in ACK1, pY99-H3 and SLC6A4 proteins levels (FIG. 4C). Similarly, pancreas isolated from KO mice too revealed loss of pY99-H3 and SLC6A4 levels, as compared to WT pancreas (FIG. 4D).

MEFs were generated from WT and KO mice; immunoblotting of lysates made from MEF too revealed significant loss of ACK1 and pY99-H3 in KO MEFs (FIG. 4E).

ACK1/pY99-H3/SLC6A4 signaling in human JAR cells expressing SLC6A4

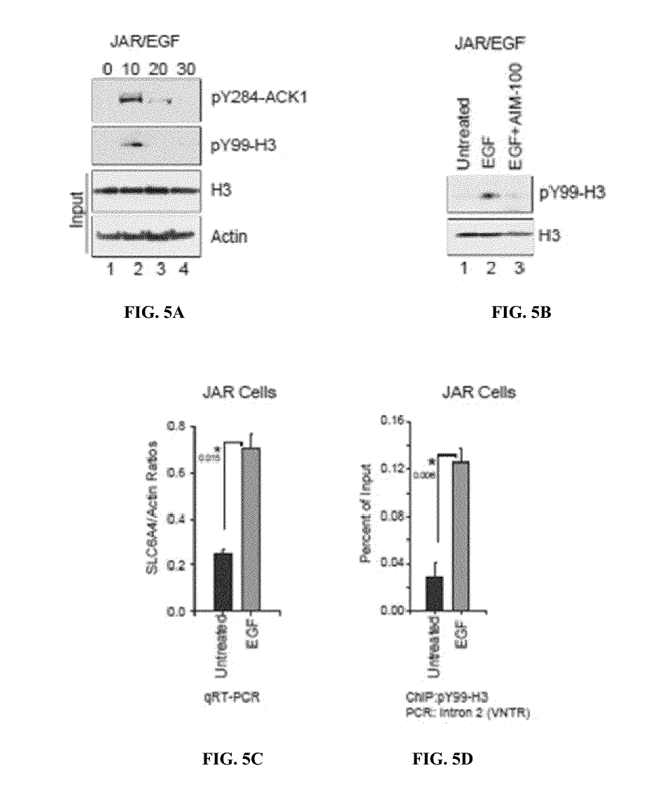

JAR cells are human placenta-derived epithelial cells that express SLC6A4 or SERT. Applicant first assessed presence of ACK1 signaling in JAR cells by treating these cells with EGF ligand. EGF ligand caused robust ACK1 activation, as seen by its phosphorylation at 10 min of treatment (FIG. 5A, top panel). ACK1 activation was also reflected in histone H3 Tyr99-phosphorylation (FIG. 5A, 2^{nd} panel).

To validate critical role of ACK1 in histone H3 Tyr99-phosphoryation, JAR cells were treated with EGF or EGF+AIM-100. EGF treatment resulted in significant upregulation of histone H3 Tyr99-phosphoryation, however, treatment with ACK1 inhibitor AIM-100 resulted in almost complete loss of histone phosphorylation (FIG. 5B).

To assess role of ACK1 in regulating SLC6A4 transcription, JAR cells were treated with EGF ligand to activate ACK1, followed by RNA preparation. The real time RT-PCR revealed significant increase in SLC64A mRNA levels upon ACK1 activation (FIG. 5C).

ACK1 Deposits pY99-H3 Marks in SLC6A4 Promoter (5-HTTLPR) and Intron 2 (VNTR)

5-HTTLPR (serotonin transporter linked polymorphic region) is a degenerate repeat polymorphic region present in the promoter region of the SLC6A4 gene. This region and its polymorphism has been extensively investigated in connection with neuropsychiatric disorders. Researchers commonly report it with two variations in humans: A short (“s”) and a long (“1”), but it can be subdivided further. In connection with the region are two single nucleotide polymorphisms (SNP): rs25531 and rs25532. The polymorphism of this repetitive element provides evidence for allele-dependent differential 5-HTT promoter activity. Allelic may play a role in the expression and modulation of complex traits and behavior. A repeat length polymorphism in the promoter of this gene has been shown to affect the rate of serotonin uptake and may play a role in sudden infant death syndrome, aggressive behavior in Alzheimer disease patients, and depression-susceptibility in people experiencing emotional trauma.

Another polymorphism seen in SLC6A4 gene is the STin2 (intron 2) VNTR, which involves different alleles that correspond to 12-, 10-, 9-, or 7-repeat units of 17 bp. VNTR polymorphism has also been associated in some cases with obsessive-compulsive disorder (OCD). The STin2.12 carriers were reported to be at over 3× risk of OCD based on a study of ˜100 OCD patients. The efficacy of commonly prescribed antidepressant drugs, such as paroxetine, has also been linked to SLC6A4 VNTR variants.

Applicant hypothesized that ACK1 deposits pY99-H3 epigenetic marks in promoter (5-HTTLPR) and intron 2 (VNTR) of SLC6A4 gene to regulate its transcription. To test this hypothesis, applicant performed chromatin immunoprecipitation (ChIP) with pY99-H3 antibodies followed by real time PCR for primers corresponding to promoter (5-HTTLPR) and intron 2 (VNTR) of SLC6A4 gene. EGF treatment of JAR cells resulted in significant increase in deposition of pY99-H3 marks at promoter (5-HTTLPR) and intron 2 (VNTR) of SLC6A4 gene (FIG. 5D).

### ACK1 KO Mice Behavioral Studies

To determine role of ACK1 epigentic signaling on SLC6A4 mRNA expression and its physiological outcome, Applicant assessed ACK1 KO mice behaviour. Totally 36 mice were used in this study, 9 Wt males, 9 KO males, 9 Wt females and 9 KO females). All the animals were age matched.

Behavioral characterization involves an initial testing of behaviors involving normal somatosensory ability and evaluation of general activity and coordination. ACK1 KO mice have normal motor coordination and learning as assessed with the accelerating rotorod test). Overall locomotor activity in the open field test and muscle strength determined with the front limb grip test revealed no abnormalities. Finally, the hot plate test showed no defects in nociception or the animal's ability to sense pain. The absence of behavioral abnormalities in these tests are ultimately important when assessing nearly all other behavioral tests.

The elevated plus maze test showed an increase in anxiety behavior in ACK1 KO mice; however, this was only significant for male ACK1 KO mice tested. In addition, a significance increase in compulsive behavior was seen in the marble burying test in both sexes of ACK1 KO mice, showing significantly increased number of marbles buried. Aggressive or dominant behavior was increased as well in the ACK1 KO mice determined by the tube test. Taken together, these tests suggest that ACK1 KO mice have a higher general anxiety coupled with aggressive behavior, which show a possible genotype/gender interaction.

Learning and memory is also affected by the loss of ACK1. This is demonstrated by a significant disruption in spatial learning and memory assessed by an increase in latency and error to find a hidden platform in the reversal radial arm water maze. Synaptic plasticity was evaluated with field recordings of Area CAl in the hippocampus. ACK1 KO mice show normal synaptic transmission, but impaired hippocampal long-term potentiation. The defect in synaptic plasticity supports the hypothesis that ACK1 deficiency disrupts cognitive function. The anxiety and aggression phenotype suggests that this synaptic alteration extends past the hippocampus and that males are more susceptible to ACK1-dependent changes in specific behaviors.

Applicant identified a novel epigenetic marks, histone H3 phosphorylation at Tyrosine 99, deposited by ACK1. pY99-H3 specific antibodies (both monoclonal and polyclonal) were extensively studied and characterized.

Applicant also identified for the first time that human and mouse serotonin transporter (SLC6A4 or SERT or 5-HTT) gene expression is regulated by ACK1. A novel mechanism was identified that is operational in SLC6A4 gene regulation. ACK1 deposits pY99-H3 epigenetic marks in promoter (5-HTTLPR) and intron 2 (VNTR) of SLC6A4 gene to regulate its transcription. Interestingly, inhibition of ACK1 by small molecule inhibitor e.g. AIM-100 or DZ1-067 resulted in significant loss of SLC6A4 mRNA expression. Without being bound by theory, applicant believes that expression of Gabra6, Slc6a2, Slc6a4, Slc6a5 and Oxt genes as well as genes shown in Table 2 are also regulated by ACK1/pY99-H3 epigenetic activity/signaling.

Applicant's data indicates that pY99-H3 antibodies can be used as companion diagnostic, for patients with infantile-onset epilepsy, cognitive regression and cases with obsessive-compulsive disorder (OCD). Most significantly, the patients which exhibit increased pY99-H3 levels should be selected as likely responsive to ACK1 small molecule inhibitors, opening a novel treatment option for these disorders. This data also shows that that pY99-H3 epigenetic marks can be an independent markers for prescription of antidepressant therapy or therapy for cocaine abuse.

Indeed, ACK1 inhibitors could be used as a novel class of antidepressants. Further, these data opens up the possibility that ACK1 inhibitor mediated downregulation of SLC6A4 or SERT could antagonize cocaine binding to SERT and thus act as a therapeutic agents for cocaine abuse.

TABLE 2
Genes regulated by ACK1 in brain, identified by RNA sequencing

| Gene name | WT Brain | KO Brain | log2(fold_change) | p_value | q_value | significant |
|---|---|---|---|---|---|---|
| Alb | 1.94891 | 0.020322 | −6.58345 | 7.66E−05 | 0.0381868 | Yes |
| Ppp1r3g | 82.8761 | 2.24212 | −5.20802 | 4.47E−05 | 0.0261212 | Yes |
| Slc6a2 | 0.555935 | 0.017482 | −4.99095 | 9.73E−05 | 0.0462526 | Yes |
| Tph2 | 6.32085 | 0.205216 | −4.9449 | 1.35E−10 | 6.87E−07 | Yes |
| Slc6a4 | 3.33809 | 0.116678 | −4.83842 | 3.22E−06 | 0.00313617 | Yes |
| Gata3 | 0.899524 | 0.043877 | −4.35763 | 0.0001008 | 0.0468263 | Yes |
| Glra1 | 5.70923 | 0.577512 | −3.30537 | 6.23E−06 | 0.00578814 | Yes |
| Ack1, Tnk2 | 6.3184 | 0.734854 | −3.10403 | 7.33E−05 | 0.0374607 | Yes |
| Calca | 8.46518 | 1.1076 | −2.9341 | 5.52E−05 | 0.0290119 | Yes |
| Slc6a5 | 12.9346 | 1.90343 | −2.76457 | 2.52E−10 | 1.03E−06 | Yes |
| Sncg | 77.0759 | 12.5702 | −2.61627 | 1.78E−07 | 0.00028024 | Yes |
| Irx2 | 4.16605 | 0.702331 | −2.56846 | 8.65E−05 | 0.042107 | Yes |
| Hbb-bt | 524.27 | 114.233 | −2.19834 | 2.26E−07 | 0.00032918 | Yes |
| Spp1 | 45.219 | 10.3469 | −2.12773 | 1.65E−06 | 0.00198129 | Yes |
| Hba-a2, Hba-a1 | 738.114 | 180.716 | −2.03012 | 2.20E−06 | 0.00249545 | Yes |
| Hba-a2, Hba-a1 | 835.76 | 208.738 | −2.00139 | 3.17E−06 | 0.00313617 | Yes |
| Gm16532 | 5.24429 | 1.46814 | −1.83676 | 4.13E−05 | 0.0248033 | Yes |
| Ret | 4.76122 | 1.33469 | −1.83482 | 5.54E−05 | 0.0290119 | Yes |
| Slc17a6 | 6.79076 | 2.09126 | −1.6992 | 5.44E−05 | 0.0290119 | Yes |
| Ppp1r1b | 8.83961 | 31.4455 | 1.8308 | 4.80E−05 | 0.0272398 | Yes |
| Pigr | 4.20038 | 15.5143 | 1.885 | 6.97E−06 | 0.00619683 | Yes |
| C4b | 2.80619 | 10.446 | 1.89627 | 2.95E−05 | 0.0197731 | Yes |
| Neurod1 | 1.95614 | 7.49857 | 1.9386 | 2.43E−05 | 0.0180222 | Yes |
| Crym | 9.91608 | 47.5086 | 2.26035 | 9.39E−07 | 0.00119958 | Yes |
| Lyz2 | 3.74131 | 18.3217 | 2.29193 | 2.04E−05 | 0.0160355 | Yes |
| Drd1a | 0.643502 | 3.15974 | 2.29579 | 3.00E−05 | 0.0197731 | Yes |
| Car8 | 0.41701 | 2.69036 | 2.68965 | 1.11E−07 | 0.00020578 | Yes |
| Car12 | 0.352329 | 2.58188 | 2.87343 | 1.13E−05 | 0.00958353 | Yes |
| Six3 | 0.244286 | 1.99032 | 3.02636 | 3.76E−05 | 0.0232849 | Yes |
| Clic6 | 0.194577 | 1.78913 | 3.20084 | 3.72E−05 | 0.0232849 | Yes |
| Ppp1r17 | 0.605376 | 7.00064 | 3.53158 | 3.63E−08 | 7.42E−05 | Yes |
| Cbln3 | 0.642005 | 8.09188 | 3.65582 | 1.19E−12 | 8.11E−09 | Yes |
| Gabra6 | 0.537633 | 6.78871 | 3.65844 | 8.42E−10 | 2.87E−06 | Yes |
| Barhl2 | 0.12421 | 1.72216 | 3.79337 | 1.62E−05 | 0.0132722 | Yes |
| Oxt | 2.92066 | 86.7988 | 4.89331 | 1.35E−07 | 0.00022993 | Yes |
| Il20rb | 0.068924 | 2.18908 | 4.98917 | 2.90E−05 | 0.0197731 | Yes |
| Cst7 | 0.155665 | 6.63763 | 5.41415 | 2.47E−05 | 0.0180222 | Yes |
| Ttr | 18.8123 | 867.438 | 5.52701 | 0 | 0 | Yes |
| Erdr1 | 0.819535 | 175.578 | 7.74309 | 0 | 0 | Yes |

### Example 3

### Role of Histone H4 Tyrosine 88 Phosphorylation in Castration Resistant Prostate Cancer

Androgen receptor (AR) plays a paramount role in the onset and progression of prostate cancer (PC) (Burnstein, K. L. (2005) Journal of Cellular Biochemistry 95(4):657-669; Chen, C. D. et al. (2004) Nature Medicine 10(1):33-39). This very facet underlies androgen deprivation therapy (ADT), a preferred treatment to negate AR transcriptional co-activator activity for advanced PC. While chemical treatment with AR antagonists or surgical treatment by orchiectomy provides immediate palliative benefits, these ADTs are ineffective long term, as the recalcitrant disease recurs within 2-3 years. Consequently, resistance to ADT has become one of the most vexing problems in prostate cancer therapy (Burnstein, K. L. (2005) Journal of Cellular Biochemistry 95(4):657-669; Feldman B. J. et al. (2001) Nature Reviews 1(1):34-45). Moreover, a majority of the PC patients' progress to a lethal stage of the disease, referred to as the Castration Resistant Prostate Cancer (CRPC). In a significant number of human CRPCs, AR mRNA is upregulated, suggesting that PC cells have reinvigorated AR transcription as a response to the loss of existing AR activity by ADT. Despite intensive efforts, targeting factors that regulate AR mRNA expression efficaciously with small molecule inhibitors has not been achieved.

Applicant has demonstrated for the first time that ACK1 tyrosine kinase interacts with AR, modifies it by tyrosine phosphorylation (pY267-AR) and this ACK1/pY267-AR complex is targeted to the chromatin to regulate AR-dependent gene expression in PC cells (Mahajan, N. P. et al. (2007) Proceedings of the National Academy of Sciences of the United States of America 104(20):8438-8443; Mahajan, N. P. et al. (2005) Cancer Research 65(22):10514-10523; Mahajan, K. et al. (2010) PloS One 5(3):e9646; Mahajan, K. et al. (2010) Journal of Cellular Physiology 224(2):327-333; Mahajan, K. et al. (2012) The Journal of Biological Chemistry 287(26):22112-22122). A critical role for ACK1 in PC pathogenesis is further underscored by several observations; namely, ACK1 mRNA is not only upregulated in prostate cancers, but activated ACK1 expression correlates positively with the progression to the malignant CRPC stage (Mahajan, N. P. et al. (2007) Proceedings of the National Academy of Sciences of the United States of America 104(20):8438-8443; Mahajan, K. et al. (2010) The Prostate 70(12):1274-1285). Indeed, 10 out 13 CRPC tumors exhibited 5- to >100-fold ACK1 overexpression (van der Horst, E. H. et al. (2005) Proceedings of the National Academy of Sciences of the United States of America 102(44):15901-15906). Consistently, LNCaP cells expressing activated ACK1, formed robust xenograft tumors in castrated nude male mice (Mahajan, N. P. et al. (2007) Proceedings of the National Academy of Sciences of the United States of America 104(20):8438-8443; Mahajan, K. et al. (2012) The Journal of Biological Chemistry 287(26):22112-22122). Furthermore, transgenic mice expressing activated ACK1 in prostates develop prostatic intraepithelial neoplasia (mPINs) and rare carcinomas (Mahajan, K. et al. (2010) PloS One 5(3):e9646; Mahajan, K. et al. (2012) The Journal of Biological Chemistry 287(26):22112-22122). Notably, alterations in ACK1 expression is associated with median disease free state of only 1.3 months compared to 110 months for PC patients without ACK1 alteration (cBioPortal). These findings underscore a dominant role for ACK1 in hormone refractory PC.

Towards Dissecting the Mechanism by which ACK1 Promotes CRPC Progression

This work has led to two significant observations. First, ACK1 regulated AR transcription directly in multiple PC cell lines. Second, ACK1 modified the chromatin via phosphorylation of histone H4 at a novel site, tyrosine 88 (pY88-H4). Importantly, the pY88-H4 epigenetic marks were deposited within the AR gene itself in an androgen-independent manner. Strikingly, reversal of this pY88-H4 histone modification, attained by ACK1 inhibition, significantly suppressed AR transcription. Moreover, this data reveal that WDR5/MLL2, members of the histone-Lysine N-Methyltransferase complex (Shahbazian, M. D. et al. (2007) Annual Review of Biochemistry 76:75-100; Shilatifard, A. (2008) Current Opinion in Cell Biology 20(3):341-348), interact with the pY88-H4 epigenetic marks, revealing a novel mode of AR epigenetic regulation. Applicant demonstrated that neoplastic PC cells adapt rapidly to ADT by recruiting the AR/ACK1 complex to the AR gene. In this androgen-deprived milieu, ACK1 catalyzes Y88-H4 phosphorylation that in turn recruits the chromatin remodeling protein WDR5, to stimulate AR transcription and facilitate CRPC development.

### ACK1 Kinase Activity Required for AR Expression in Androgen Deficient Environment

CRPC remains an incurable malignancy with limited treatment options and is a significant cause of deaths in men—both US and worldwide (Greenlee, R. T. et al. (2000) CA: A Cancer Journal for Clinicians 50(1):7-33). Androgen receptor signaling is found to be operational pre- and post-castration stage, albeit disparate mechanisms operate in PC cells to promote androgen dependent and independent AR transcriptional co-activator activity. These distinct AR regulatory activities are manifested as distinct transcriptional programs operational in PC cells that contribute favorably towards cell survival, proliferation and growth. Not surprisingly, AR protein has been the epicenter of targeted therapeutic approaches. Recently, Enzalutamide or MDV3100 (marketed as Xtandi), an AR antagonist, has been FDA approved for treatment of metastatic CRPC patients (Tran, C. et al. (2009) Science 324(5928):787-790). Although highly effective in suppressing AR activity, and also nuclear translocation (seen by significant decrease in serum PSA levels), it is effective only in a subset of CRPC patients (12 out of 30 patients) (Tran, C. et al. (2009) Science 324(5928):787-790). Moreover, the overall survival advantage was found to be modest (4-6 months) and even the most responding patients relapsed within ˜2 years (Bennett, L. L. et al. (2014) The Annals of Pharmacotherapy 48(4):530-537). Interestingly, these relapsed patients exhibit renewed AR target gene expression by multiple mechanisms, suggesting that CRPC has bypassed Enzalutamide blockade (Balbas, M. D. et al. (2013) eLife 2:e00499; Arora, V. K. et al. (2013) Cell 155(6):1309-1322; Joseph, J. D. et al. (2013) Cancer Discovery 3(9):1020-1029). These setbacks revealed two major caveats of tackling this complex disease; first, not all CRPCs are the same and second, other signaling events may be driving the disease, which explains the efficacy of Enzalutamide in a limited number of CRPC patients. Moreover, because CRPCs display de novo or intrinsic ability to increase AR levels, inhibition of AR protein activity is not enough (Knuuttila, M. et al. (2014) Am. J. Pathol). To achieve complete remission, ablation of AR transcription appears to be the key for all AR-dependent prostate cancers. However, targeted inhibition of AR transcription with small molecule inhibitors has not yet been accomplished.

Transcriptional regulation of the AR gene itself is a paradigm that merits thorough investigation. Epigenetic modifications are intricately linked to transcription events, especially when activated by nuclear hormones (Xu, K. et al. (2012) Science 338(6113):1465-1469; Cai, C. et al. (2011) Cancer Cell 20(4):457-471). The data obtained has indicated that ACK1 kinase is a unique tyrosine kinase that not only binds tightly to AR in androgen-deficient environment, but also ‘piggybacks’ AR to the nucleus to bind chromatin (Mahajan, N. P. et al. (2007) Proceedings of the National Academy of Sciences of the United States of America 104(20):8438-8443). Whether AR utilizes ACK1 to facilitate its transcriptional co-activator function is not known. Towards understanding the outcome of the androgen-independent AR/ACK1 cross talk, an unbiased approach was selected wherein androgen-deprived prostate cancer cells LNCaP were treated with dihydrotestosterone (DHT) or Enzalutamide.

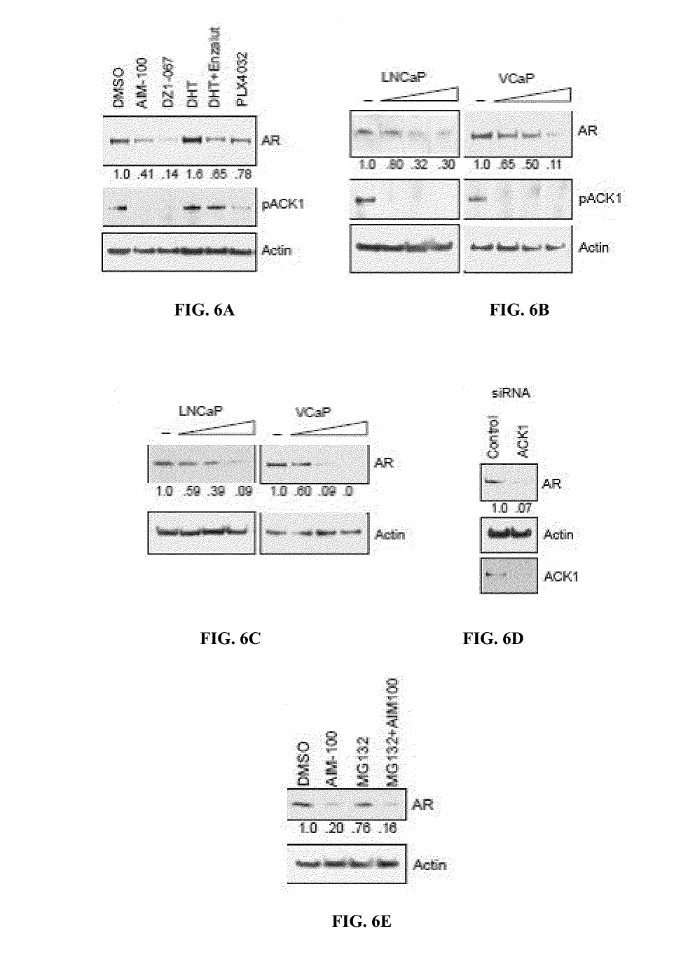

To assess whether loss of AR levels is dependent on specific inhibition of ACK1 kinase activity, increasing concentrations of AIM-100 were used. A concomitant decrease in AR protein levels was observed which correlated with increasing amounts of AIM-100 in two different PC lines, LNCaP and VCaP cells (FIG. 6B), suggesting that ACK1 kinase activity is critical for maintaining AR levels in androgen-deficient environment of prostate cancer cells.

Similarly, increasing concentrations of DZ1-067 were used. A concomitant decrease in AR protein levels was observed which correlated with increasing amounts of DZ 1-067 in two different PC lines; LNCaP and VCaP cells (FIG. 6C),

To examine that loss of AR levels is not due to ‘off target effect’ of ACK1 inhibitors, LNCaP cells were transfected with ACK1 siRNA. Immunoblotting revealed significant decrease in AR levels upon loss of ACK1 (FIG. 6D).

### ACK1 Mediated Loss of AR Expression is not Due to Proteasome-Dependent Degradation.

AR has been known to interact with an ubiquitin E3 ligase, RNF6, causing AR ubiquitination, which in turn promoted AR transcriptional activity (Xu, K. et al. (2009) Cancer Cell 15(4):270-282). To determine whether post-translational modification has role to play in suppression of AR levels upon ACK1 kinase inhibition, LNCaP cells were treated with proteosomal inhibitor, MG-132 and AR levels were measured in presence or absence of AIM-100. Proteosomal inhibitor did not prevent loss of AR caused by ACK1 kinase inhibition, suggesting that ACK1 regulates AR levels at transcriptional stage.

ACK1 Kinase Activity Required for Restoring AR mRNA Levels in Androgen Deficient Environment

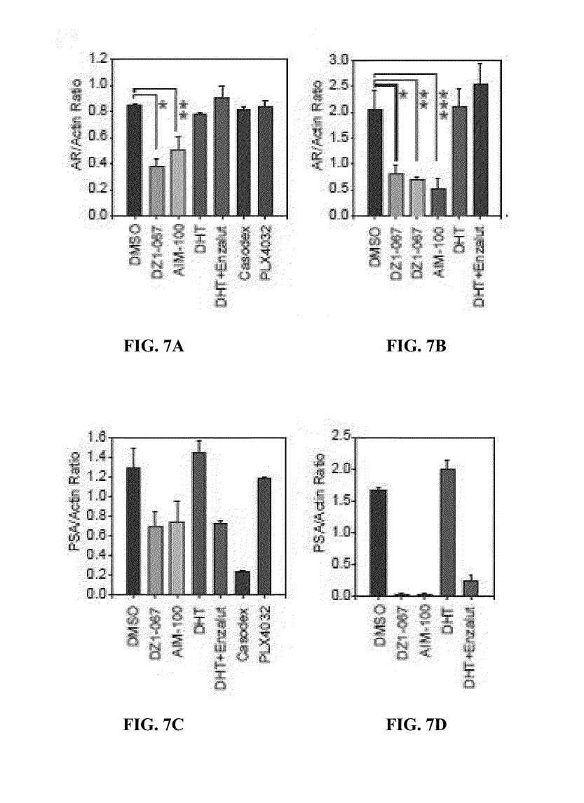

To validate this data further, androgen-deprived LAPC4 and LNCaP cells were treated with DZ1-067, AIM-100, DHT, Enzalutamide, Casodex or PLX4032. Total RNA was isolated followed by real time PCR, which revealed that AR mRNA levels were significantly suppressed upon DZ1-067 or AIM-100 treatment, however, no significant change in AR mRNA levels were seen upon DHT, Enzalutamide, Casodex or PLX4032 treatments (FIGS. 7A and 7B). Prostate specific antigen (PSA) is a major AR target gene whose expression reflects AR transcriptional co-activator ability, too exhibited significant loss upon ACK1 inhibitor treatment (FIGS. 7C and 7D). Interestingly, first generation (Casodex) and second generation (Enzalutamide) of anti-androgens although did not overturn AR mRNA levels, significantly suppressed PSA mRNA levels, as reported in literature (FIGS. 7C and 7D). Taken together, these data indicate that ACK1 kinase plays a crucial role in maintaining AR mRNA levels, in absence of androgen, by regulating its transcription.

ACK1 kinase activity required for prostate cancer cell proliferation in androgen deficient environment.

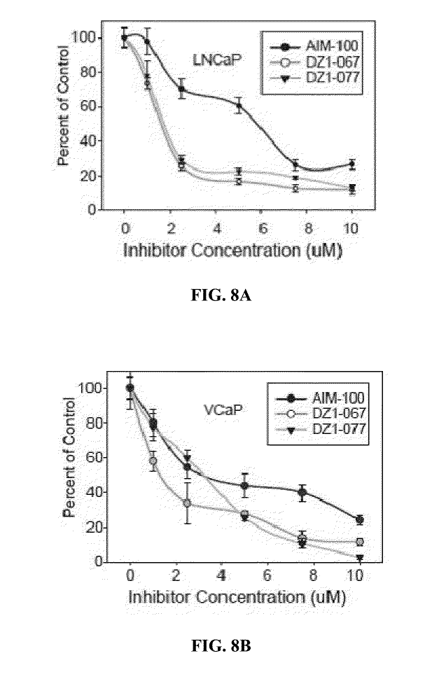

Applicant also assessed ability of the new ACK1 inhibitors to suppress proliferation of prostate cancer cell lines. Both, DZ1-067 and DZ1-077 were significantly better (IC₅₀=1.8 uM) than AIM-100 (IC₅₀=7 uM) in their ability to inhibit cell growth in LNCaP cells (FIG. 8A). In contrast, androgen-independent VCaP cells were observed to be highly sensitive to DZ1-067 (IC₅₀=2 uM), while AIM-100 and DZ1-077 exhibited IC₅₀ of 4 uM. Overall, it appears that ACK1 is needed for androgen-independent growth of prostate cancer cells. And that is why, DZ1-067, an excellent ACK1 inhibitor exhibit significant potential to suppress proliferation of androgen-independent or CRPC cells.

ACK1 Expressing Prostate Cancers Patients with Low Disease-Free Survival.

A larger cohort of data has recently become available at cBioPortal. Of the 216 patients with prostate adenocarcinoma, 33 patients with high ACK1 mRNA expression or mutation exhibited median disease free survival of 1.38 months and 10 cases exhibited a relapse. In contrast, those patients that did not have alterations in ACK1 had significantly longer disease free survival (110.33 months) and 50 cases relapsed.

These data suggests that the fraction of prostate cancer patients that have aberrant ACK1 expression are likely to rapidly progress to CRPC, a major cause of death. Interestingly, ACK1 alteration and AR gene amplification or mutation had no co-relation (Odds Ratio: 1.36; 95% Confidence Interval: 0.54-3.43; Fisher's Exact Test p-value: 0.32), suggesting that ACK1 mediated AR transcriptional upregulation is an independent mechanism.

### A Novel Function of ACK1 as a Histone Tyrosine Kinase.

Although, Applicant's prior research has established a crucial role of ACK1 in progression of prostate cancer to CRPC stage, the exact molecular processes executed by ACK1 to cause the dramatic shift in AR activity as well as its mRNA/protein levels are essentially unknown. Applicant reasoned that AR-Bound ACK1 could potentially interact with the chromatin, especially histones and could modify them to generate favorable epigenetic atmosphere. Being a tyrosine kinase, ACK1 could phosphorylate histone at tyrosine residue, however, when Applicant initiated this study, the phenomenon of histone tyrosine phosphorylation was not reported in the literature. Sensing an opportunity that could have considerable significance, histone purification followed by mass spectrometry was performed, which lead to the identification of a novel histone phosphorylation events, phosphorylation of histone H4 at Tyr88 (pY88-H4).

The functional role of pY88-H4 epigenetic marks and especially the kinase that could bring about this modification was not clear. Applicant assessed whether ACK1 is the kinase that can phosphorylate H4 by transfecting HEK293 cells with activated ACK1, followed by mass spectrometry analysis of purified histones. It revealed that histone H4 is phosphorylated at Tyr88 residue by ACK1.

Generation of pY88-H4 Specific Monoclonal Antibodies.

To assess the functional role of the epigenetic modification, mouse monoclonal antibodies were generated that specifically recognize pY88-H4 marks. In brief, two peptides coupled to immunogenic carrier proteins were synthesized (Ac-K-Ahx-RKTVTAMDVVpYALKR; Ac-K-Ahx-RKTVTAMDVVYALKR-amide). Two mice were immunized with the phosphopeptide. About 100 clones were checked by ELISA and 8 clones that exhibited binding exclusively to phosphopeptide were then used for western analysis. Two clones (A2 and A9) were found to express antibodies that exclusively recognized Tyr88-phosphorylated histone H4 (or pY88-H4). These antibodies were affinity-purified and validated by dot blot analysis.

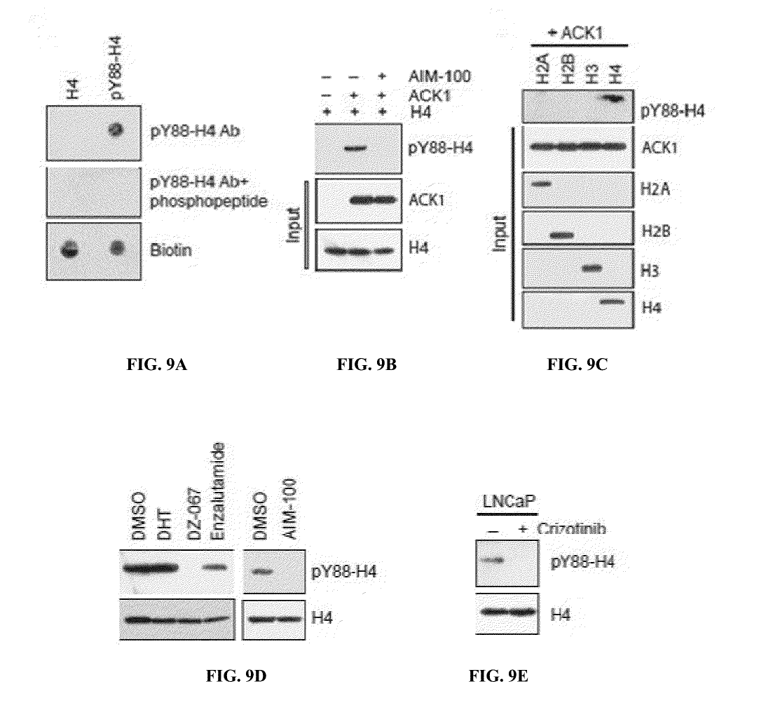

Biotinylated phosphopeptide and identical but unmodified peptide was spotted on nitrocellulose membrane followed by immunoblotting with pY88-H4 antibody. The pY88-H2B antibody specifically recognized the phosphorylated peptide but failed to recognize the unphosphorylated peptide (FIG. 9A, top panel). To further evaluate specificity of antibodies, dot blot with phospho and unmodified peptide were immunoblotted with pY88-H2B antibody that was pre-incubated with the phosphopeptide RKTVTAMDVVpYALKR. The phosphopeptide competed with pY88-H2B antibody for binding to phosphopeptide that has been spotted on the filter, dampening the signal (FIG. 9A).

### ACK1 Directly Binds and Phosphorylates Histone H4 at Tyrosine 88.

To assess direct binding of ACK1 to H4, in vitro kinase assay was performed using purified ACK1 and H4 (New England Biolabs). Human ACK1 were expressed in insect cells and purified to homogeneity (unpublished data). Immunoblotting with pY88-H4 and pTyr antibodies confirmed that indeed H4 is directly Tyr-phosphorylated by ACK1 (FIG. 9B). Further, H4 Tyr-phosphorylation is abrogated by treatment with ACK1 inhibitor, AIM-100 (FIG. 9B), suggesting that ACK1 directly binds and phosphorylates histone H4.

To further assess the specific ACK1-H4 interaction in vitro kinase assay was performed wherein purified ACK1 was incubated with all the four core histones, followed by immunoblotting with pY88-H4 antibodies. Presence of band exclusively when ACK1 was incubated with H4 indicates specificity of pY88-H4 antibodies (FIG. 9C).

### ACK1 Phosphorylates Endogenous Histone H4 at Tyrosine 88.

To explore the sensitivity of endogenous H4 pY88-phosphorylation to ACK1 inhibitor, LNCaP cells were treated with DZ1-067, AIM-100 or Enzalutamide. LNCaP cells exhibited robust expression of endogenous pY88-phosphorylation of H4, which was eliminated upon treatment with DZ1-067, but was unaffected by DHT or Enzalutamide.

Applicant also assessed the sensitivity of endogenous H4 pY88-phosphorylation to small molecule inhibitor, Crizotinib, also known as Xalkori (Pfizer). Crizotinib is an anti-cancer drug acting as an ALK (Anaplastic Lymphoma Kinase) and ROS1 (c-Ros Oncogene 1) and have non-specific ability to inhibit ACK1. It is approved for treatment of some non-small cell lung carcinoma (NSCLC). LNCaP cells were treated with Crizotinib exhibited significant loss of endogenous pY88-phosphorylation of H4 (FIG. 9E). Collectively, these data established that ACK1 tyrosine kinase is a novel epigenetic modifier.

ACK1 deposits the pY88-H4 epigenetic marks at the AR intron 2 and 3′ UTR AR is essential for not only in normal prostate but also for malignant prostate tumor growth (Feldman, B. J. et al. (2001) Nature Reviews 1(1):34-45). The modus operandi of this hormone receptor is now conclusively established wherein androgen-bound AR initiates transcription of target genes, e.g., PSA, by binding to androgen-response elements (ARE) in promoter regions. However, this paradigm was shaken to core when CRPC tumors were found to be not only thriving under low castration levels of androgen but also maintained functional AR, suggesting that AR has ‘learned’ to deal with dwindling androgen levels. But, then how does AR mount the response when androgen is depleted and is there an assisting companion?

Interestingly, applicant observed that not only is AR/ACK1 complex bound to the chromatin in androgen-deficient environment (Mahajan, N. P. et al. (2007) Proceedings of the National Academy of Sciences of the United States of America 104(20):8438-8443), but studies revealed that ACK1 inhibitors also caused significant loss of AR transcription (FIGS. 6A-6E, 7A-7D, 8A and 8B, 9A-9E). Taken together, this data uncovers a distinct epigenetic mechanism wherein AR regulates its own transcription in androgen deficient environment by availing the chromatin modifying activity of the ACK1 kinase.

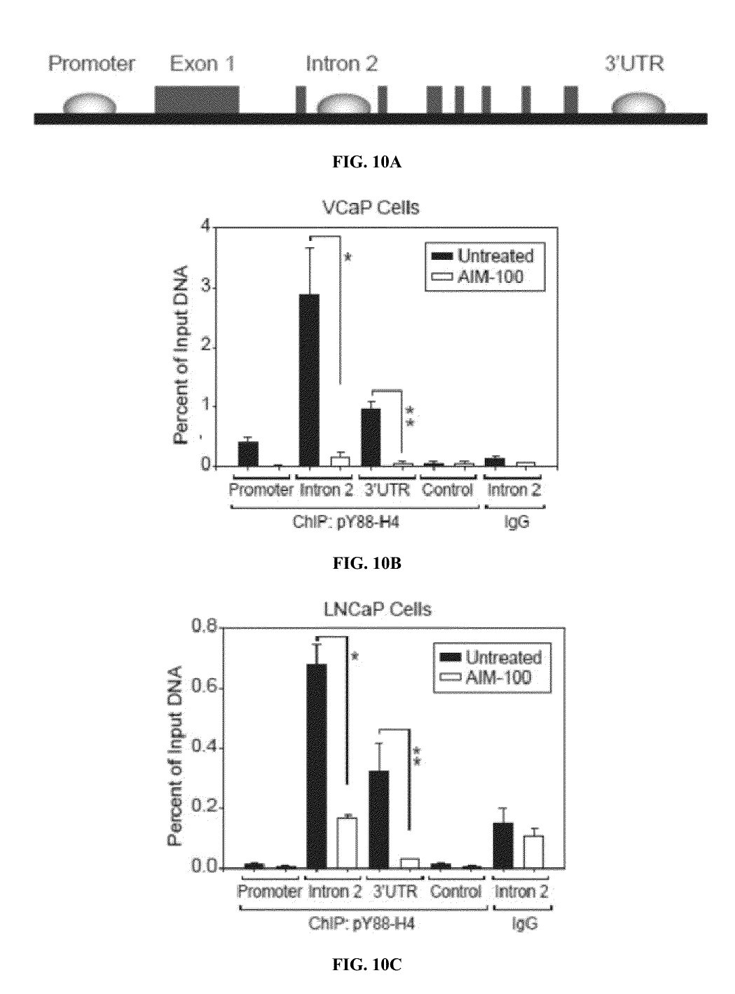

To determine whether ACK1 modifies chromatin at AR gene locus, LNCaP and VCaP cells grown in the absence of androgen and were treated with AIM-100. Chromatin immunoprecipitation (ChIP) was performed using pY88-H4 antibodies, followed by real time PCR with primers corresponding to the AR promoter, intron 2 and 3′UTR (FIG. 10A). ChIP data revealed the presence of pY88-H4 marks predominantly at the intron 2. These marks were also deposited at 3′UTR of the AR gene but not at the promoter region (FIGS. 10B and 10C). Significantly, these epigenetic marks were erased in AIM-100 treated samples, suggesting that deposition of pY88-H4 epigenetic marks in AR gene is a reversible event and can be accomplished using ACK1 inhibitors.

Collectively, the data reveal the role of a novel chromatin alteration event, histone H4 tyrosine phosphorylation mediated by the oncogenic kinase ACK1, as a critical factor driving AR mRNA expression in CRPC.

Characterization of this novel mode of AR epigenetic regulation which facilitates a continuum of AR expression and AR dependent transcriptional program, despite ADT and determining how ACK1 small molecule inhibitors shut down AR transcription to cause death of the recalcitrant CRPCs will be a definitive step to develop effective therapies in clinical setting.

### Generation of Isogenic Human Prostate Cancer Model Cell Lines Harboring ACK1 Genetic Deletion Using the CRISPR/Cas9 Technology to Characterize its Role in the CRPCs.

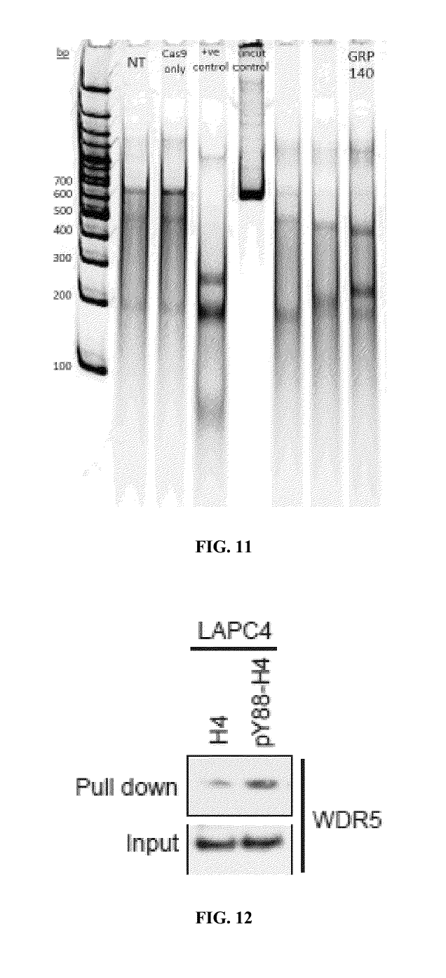

The clustered regulatory interspaced short palindromic repeats (CRISPR) nuclease system has revolutionized the way one can edit and engineer the genomic locus of choice, in a highly sequence-specific manner (Jinek, M. et al. (2012) Science 337(6096):816-821). It takes advantage of a short guide RNA (gRNA) to target the bacterial Cas9 endonuclease to specific genomic loci. Applicant has generated three different ACK1 gRNA constructs which were validated by transfecting HEK293 cells, followed by PCR using primers spanning cleavage site in ACK1 (F: CCGTGTAGTGGGATGAAGGT (SEQ ID NO: 13) and R: AAGAGAGCGTGAGCACGAAT (SEQ ID NO: 14). The 681 bp fragment was heated and re-annealed to form heterodimers which were digested with Cel-I enzyme to detect mismatches. Clone GRP140 (FIG. 11, last lane) shows the robust ACK1 specific cutting in the genomic locus; 681 bp band has almost completely disappeared and two bands corresponding to cleavage (440 and 240 bp bands) are clearly visible.

To delineate the pathogenic role of ACK1 signaling networks in CRPC development and progression to metastasis, Applicant transfected LNCaP and VCaP cells with ACK1 specific gRNA (seq: GTACGTCAAGAATGAGGACC)(SEQ ID NO: 15) plated in single cell dilution in a 96 well plate. The clones were grown and the ACK1 locus specific indels (generated during cellular NHEJ repair, which often cause frameshift) were detected using PCR amplification, as described above.

The Functional Role pY88-H4 Dependent Recruitment of WDRY5/MLL2 Chromatin Remodeling Complex at the AR Locus.

Epigenetic signaling networks ensure timely gene expression profiles; not surprisingly, perturbations in epigenetic signaling are a recurrent feature in cancer, hereditary and metabolic diseases. Epigenetic marks regulate transcriptionally activating or suppressive programs depending on the chromatin context. They achieve this by interacting with proteins referred to as the ‘readers’ that recognize the specific epigenetic marks, which in turn may selectively recruit chromatin altering ‘writers’ that modify the chromatin to promote a transcriptionally permissive environment. This data indicates that AR locus is a well-attended ‘field’ where several chromatin remodeling proteins ply their trade, underscoring a pathogenic role for these ‘players’ in AR expression and CRPC progression.

Identification of MLL2/WDR5 as pY88-H4 Interacting Chromatin Remodeling Complex.

To elucidate the molecular mechanism by which ACK1 mediated H4 Y88-phosphorylation regulates AR transcription, Applicant first performed an unbiased pull down to uncover readers of the pY88-H4 marks, as described in Applicant's earlier publication (Mahajan, K. et al. (2012) Nature Structural & Molecular Biology 19(9):930-937). LNCaP cells extract was incubated with biotinylated H4-Y88 phosphopeptides (unphosphorylated peptide as control) and bound proteins were analyzed by LC-MS/MS analysis. The top ‘hits’ in the Proteomic analysis turned out to be the key members of the MLL2/WDR5 chromatin remodeling complex (unpublished data).

MLL2 has H3K4 methyl transferase activity and associated regulatory protein, WDR5 recognizes the dimethyl-H3K4 marks and facilitate the conversion to tri-methyl H3K4 (H3K4me3) by the MLL methyl transferase (Wysocka, J. et al. (2005) Cell 121(6):859-872). Interestingly, H3K4me3 modification is associated with transcriptional activation in a number of contexts (Wysocka, J. et al. (2005) Cell 121(6):859-872), providing a vital clue that pY88-H4 may operate in trans to drive AR transcription.

Towards verifying these interactions, applicant first analyzed the peptide pull down followed by immunoblotting. Consistent with the proteomic analysis, WDR5 showed preferential binding to the phosphorylated Y88-H4 compared to the unphosphorylated H4 (FIG. 12A). Overall, these preliminary studies reveal for the first time that recruitment of MLL2/WDR5 complex is modulated by pY88-H4 epigenetic mark.

Recruitment of AR and Deposition of H3K4Me3 Epigenetic Marks within Intron 2 of AR Gene.

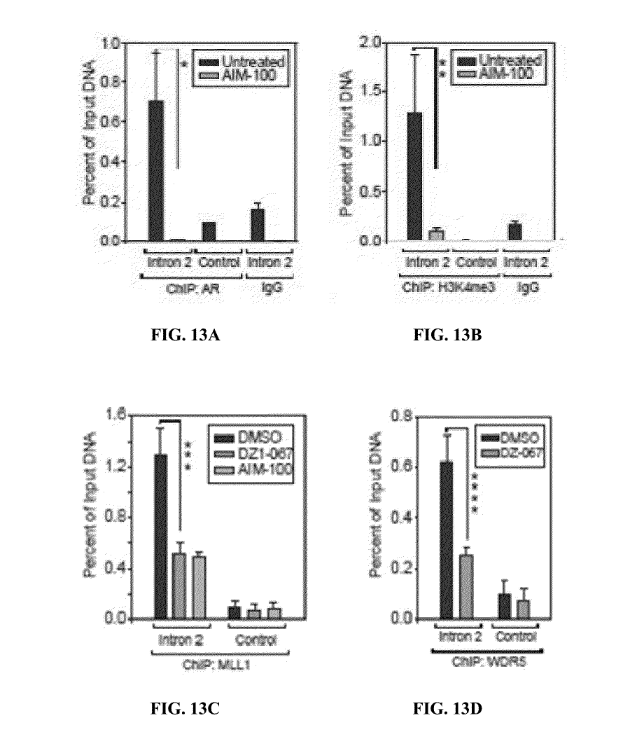

To determine the functional and physiological relevance of pY88-H4/WDR5 interaction, and given the interaction of AR with MLL associated complex (Grasso, C. S. et al. (2012) Nature 487(7406):239-243), Applicant performed ChIP experiments to determine AR and H3K4me3 levels at the intron 2. Both, AR and H3K4me3 methyl marks were found to be specifically enriched at the intron 2 region, in absence of androgen, that were abolished by treatment with AIM-100 (FIGS. 13A-13D).

Based on these data and without being bound by theory, applicant submits that ACK1 is the ‘writer’ and WDR5 is the ‘reader’ for pY88-H4 epigenetic marks. Further, MLL2 acts as the second writer′ that introduces H3K4me3 activating marks in response to action of first writer, ACK1, to drive AR expression in androgen-deficient environment of CRPC tumors.

pY88-H4 and AR Expression in Human Prostate Cancer.

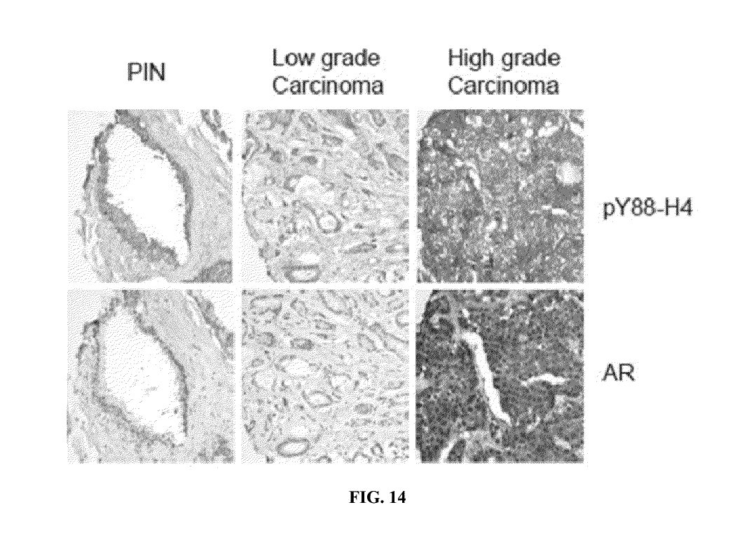

Tissue Micro Array (TMA) sections representing different prostate cancer stages stained with pY88-H4 and AR Abs. Both, pY88-H4 and AR expression increased as disease proressed to later stages of cancer (FIG. 14).

Applicant observed that pY88-H4 epigenetic footprint at AR locus was unaffected by androgen or anti-androgens, however, ACK1 inhibitors not only inhibited H4 Tyr88-phosphorylation, but also able to suppress AR transcription. Therefore, targeting ACK1 kinase in CRPC patients could prove to be a highly effective strategy to downregulate AR, the lifeline of these advanced stage prostate cancers Inhibition of ACK1 would suppress AR levels in vivo, compromising CRPC tumor growth, thus ACK1 inhibitors could emerge as new therapeutic agents in CRPCs.

The majority of the prostate cancers inevitably develop castration resistant disease that has reactivated AR synthesis. How CRPCs promote AR expression has been the topic of high relevance in prostate cancer research. Applicant has uncovered a novel mechanism of transcriptional self-activation of AR gene wherein AR protein coordinates functionally with ACK1 tyrosine kinase to modify chromatin within the AR gene. This proposal have demonstrated for the first time the role of the novel ACK1/AR epigenetic signaling nexus in CRPC progression.

While signaling mechanisms and cytosolic effectors of most tyrosine kinases have been well-studied, direct chromatin tyrosine phosphorylation in CRPC pathogenesis is unexplored. Applicant identified a previously unknown histone phosphorylation event, pY88-H4. This proposal delineate for the first time, the functional consequence of this novel epigenetic event in AR transcriptional activation in androgen-deficient environment.

Applicant also identified WDR5 as a novel epigenetic reader of pY88-H4 marks that maintains AR mRNA synthesis in androgen-depleted environment by introducing a second layer of activating epigenetic marks, H3K4me3. The studies described here provides crucial insight into the realm of AR transcriptional activation in CRPCs, wherein ACK1 epigenetic activity harmonizes with AR to recruit WDR5/MLL2 methyl-transferase to reactivate AR mRNA synthesis.

Presence of elevated AR mRNA levels in spite of prolong AR antagonist treatment in CRPCs is a paradox that has mystified researchers, yet, it exposed an Achilles' heel for targeting new treatment strategies. This data indicates that since the pY88-H4 deposition is specifically upregulated in CRPCs, removal of these activating marks by ACK1 inhibitors opens up a novel therapeutic option for this essentially incurable malignancy. This proposal provides detailed mechanistic basis to for ACK1 inhibitors, including DZ1-067 as novel epigenetic inhibitors that suppress AR transcriptional activity and CRPC tumor growth.

Undoubtedly, the foremost reason for transient effectiveness of the hormone ablation therapy is the poor understanding of the molecular mechanism/s operational during disease progression to CRPC (3). This data shows that cancer progression to CRPC requires epigenetic activity ACK1 kinase indicating that those prostate cancer patients that exhibit elevated levels of pY88-H4 are likely to respond to ACK1 inhibitor. However, screening of prostate cancer patients to facilitate personalized treatment with ACK1 inhibitor is an unmet clinical need. Applicant has generated pY88-H4 specific monoclonal antibodies that detect this histone modification event in not only cell lines but also human tumor samples (FIG. 14).

The contents of the articles, patents, and patent applications, and all other documents and electronically available information mentioned or cited herein, are hereby incorporated by reference in their entirety to the same extent as if each individual publication was specifically and individually indicated to be incorporated by reference.

Applicant reserves the right to physically incorporate into this application any and all materials and information from any such articles, patents, patent applications, or other physical and electronic documents.

The disclosures illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims. Thus, it should be understood that although the present disclosure has been specifically disclosed by preferred embodiments and optional features, modification and variation of the disclosures embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein. Other embodiments are within the following claims. In addition, where features or aspects of the disclosure are described in terms of Markush groups, those skilled in the art will recognize that the disclosure is also thereby described in terms of any individual member or subgroup of members of the Markush group.

1. A method for detecting phosphorylation of a histone H2B protein at the Tyr37 residue or for identifying or selecting a cancer patient having a wildtype BRAF genotype or mutant BRAF expressing patients who have developed resistance to BRAF inhibitors for a therapy comprising a WEE1 inhibitor, comprising detecting phosphorylation of a histone H2B protein at the Tyr37 residue in a sample isolated from the patient, wherein phosphorylation of the H2B at Tyr37 residue selects or identifies the patient for the therapy and absence of phosphorylation of the histone H2B protein at the Tyr37 residue does not identify or select the patient for the therapy.

2. A method for selecting a cancer patient having a wildtype BRAF genotype or mutant BRAF expressing patients who have developed resistance to BRAF inhibitors for a therapy comprising a WEE1 inhibitor, comprising determing the expression level of WEE1 RNA or protein and IDH2 RNA or protein in a sample isolated from the patient, wherein a) overexpression of WEE1 RNA or protein and b) underexpression of IDH2 RNA or protein in the sample as compared to a control for the WEE1 protein expression and a control for the IDH2 RNA or protein, respectively, selects the patient for the therapy and neither a) nor b) does not select the patient for the therapy.

3. The method of claim 1 or 2, further comprising administering the WEE1 inhibitor therapy to the cancer patient wherein the cancer patient suffers from brain cancer (glioblastoma multiforme), breast cancer, melanoma, lung cancer, prostate cancer.

4. The method of claim 1 or 2, wherein the activation of the WEE1 protein is determined by assessing or measuring histone H2B Tyr37-phosphorylation in the sample by immunohistochemistry, immunoprecipitation, immunoblotting, ELISA or by contacting the sample with an isolated antibody that specifically recognizes SEQ ID NO: 4 (KRSRKESpYSVYVYKVL), wherein the Y(Tyr)8 residue is phosphorylated.

5. A method for detecting phosphorylation of a histone H4 protein at the Tyr88 residue or for identifying or selecting a breast cancer (including tamoxifen-resistant breast cancer) and prostate cancer (including castration resistant prostate cancer or CRPC) patients for a therapy comprising ACK1 inhibitor therapy, comprising detecting phosphorylation of a histone H4 protein at the Tyr88 residue in a sample isolated from the patient, wherein phosphorylation of histone H4 at the Tyr88 residue selects or identifies the patient for the therapy and absence of phosphorylation of the histone H4 protein at the Tyr88 residue does not identify or select the patient for the therapy.

6. The method of claim 5, further comprising administering the therapy to the patient identified or selected for the therapy.

7. The method of claim 5, wherein the detecting comprising contacting the sample with an isolated antibody that specifically recognizes SEQ ID NO: 6 (TVTAMDVVpYALKRQGRT), wherein the Y(Tyr)9 residue is phosphorylated.

8. A method for determing the level of phosphorylation of a histone H3 protein at the Tyr99 residue or for identifying or selecting a subject in need thereof for a therapy comprising an ACK1 inhibitor, comprising determing the level of phosphorylation of a histone H3 protein at the Tyr99 residue in a sample isolated from the subject, and identifying or selecting the subject for the therapy if phosphorylation of the Tyr99 residue is detected and not selecting the patient for the therapy if the phosphorylation of the Tyr99 is not detected.

9. The method of claim 8 wherein the determining comprising contacting the sample with an isolated antibody specifically recognizes SEQ ID NO: 2 (ALQEACEApYLVGLFED), wherein the Y(Tyr)9 residue is phosphorylated.

10. A composition comprising an antibody that specifically recognizes SEQ ID NO: 6 (TVTAMDVVpYALKRQGRT), wherein the Y(Tyr)9 residue is phosphorylated for use in a method for identifying or selecting a castration resistant prostate cancer (CRPC) patient for a therapy comprising an ACK1 inhibitor.

11. A composition comprising a probe or antibody that specifically recognizes SEQ ID NO: 4 (KRSRKESpYSVYVYKVL), wherein the Y(Tyr)8 residue is phosphorylated, for determining the expression level of Tyr37-phosphorylated histone H2B and thus WEE1 kinase activity in sample for use in a method for selecting a cancer patient having a wildtype BRAF genotype or patients who have developed resistance for BRAF inhibitors for a therapy comprising a WEE1 inhibitor.

12. A composition comprising a probe or antibody for determining the expression level of IDH2 RNA or protein for use in a method for selecting a cancer patient having a wildtype BRAF genotype or patients who have developed resistance for BAF inhibitors for a therapy comprising a WEE1 inhibitor.

13. A composition comprising an antibody specifically recognizes SEQ ID NO: 2 (ALQEACEApYLVGLFED), wherein the Y(Tyr)9 residue is phosphorylated for identifying or selecting a therapy comprising an ACK1 inhibitor for a subject suffereing from a disorder selected from infantile-onset epilepsy, cognitive regression, obsessive-compulsive disorder (OCD), depression, substance dependence, and cocaine dependence.


SEQUENCE LISTING (32 sequences)

SEQ ID NO: 1 - protein, 15 aa, Artificial Sequence
  Description of Artificial Sequence Synthetic peptide
KRISGLIYEETRGVL

SEQ ID NO: 2 - protein, 16 aa, Artificial Sequence
  Description of Artificial Sequence Synthetic peptide
ALQEACEAYLVGLFED

SEQ ID NO: 3 - protein, 16 aa, Artificial Sequence
  Description of Artificial Sequence Synthetic peptide
KRSRKESYSVYVYKVL

SEQ ID NO: 4 - protein, 16 aa, Artificial Sequence
  Description of Artificial Sequence Synthetic peptide
KRSRKESYSVYVYKVL

SEQ ID NO: 5 - protein, 17 aa, Artificial Sequence
  Description of Artificial Sequence Synthetic peptide
TVTAMDVVYALKRQGRT

SEQ ID NO: 6 - protein, 17 aa, Artificial Sequence
  Description of Artificial Sequence Synthetic peptide
TVTAMDVVYALKRQGRT

SEQ ID NO: 7 - protein, 15 aa, Artificial Sequence
  Description of Artificial Sequence Synthetic peptide
KRISGLIYEETRGVL

SEQ ID NO: 8 - protein, 16 aa, Artificial Sequence
  Description of Artificial Sequence Synthetic peptide
ALQEACEAYLVGLFED

SEQ ID NO: 9 - protein, 18 aa, Artificial Sequence
  Description of Artificial Sequence Synthetic peptide
KRSRKESYSVYVYKVLXC

SEQ ID NO: 10 - protein, 18 aa, Artificial Sequence
  Description of Artificial Sequence Synthetic peptide
KRSRKESYSVYVYKVLXC

SEQ ID NO: 11 - protein, 25 aa, Artificial Sequence
  Description of Artificial Sequence Synthetic peptide
DGKKRKRSRKESYSVYVYKVLKQVH

SEQ ID NO: 12 - protein, 25 aa, Artificial Sequence
  Description of Artificial Sequence Synthetic peptide
DGKKRKRSRKESYSVYVYKVLKQVH

SEQ ID NO: 13 - DNA, 20 bp, Artificial Sequence
  Description of Artificial Sequence Synthetic primer
ccgtgtagtgggatgaaggt

SEQ ID NO: 14 - DNA, 20 bp, Artificial Sequence
  Description of Artificial Sequence Synthetic primer
aagagagcgtgagcacgaat

SEQ ID NO: 15 - DNA, 20 bp, Artificial Sequence
  Description of Artificial Sequence Synthetic oligonucleotide
gtacgtcaagaatgaggacc

SEQ ID NO: 16 - protein, 16 aa, Artificial Sequence
  Description of Artificial Sequence Synthetic peptide
ALQEACEAFLVGLFED

SEQ ID NO: 17 - protein, 17 aa, Artificial Sequence
  Description of Artificial Sequence Synthetic peptide
KXRKTVTAMDVVYALKR

SEQ ID NO: 18 - protein, 17 aa, Artificial Sequence
  Description of Artificial Sequence Synthetic peptide
KXRKTVTAMDVVYALKR

SEQ ID NO: 19 - protein, 15 aa, Artificial Sequence
  Description of Artificial Sequence Synthetic peptide
RKTVTAMDVVYALKR

SEQ ID NO: 20 - protein, 16 aa, Homo sapiens
LQEACEAYLVGLFEDT

SEQ ID NO: 21 - protein, 16 aa, Homo sapiens
LQEASEAYLVGLFEDT

SEQ ID NO: 22 - protein, 16 aa, Homo sapiens
LQEASEAYLVGLFEDT

SEQ ID NO: 23 - protein, 16 aa, Bos taurus
LQEACEAYLVGLFEDT

SEQ ID NO: 24 - protein, 16 aa, Mus musculus
LQEACEAYLVGLFEDT

SEQ ID NO: 25 - protein, 16 aa, Rattus norvegicus
LQEACEAYLVGLFEDT

SEQ ID NO: 26 - protein, 16 aa, Xenopus laevis
LQEASEAYLVGLFEDT

SEQ ID NO: 27 - protein, 16 aa, Danio rerio
LQEASEAYLVGLFEDT

SEQ ID NO: 28 - protein, 16 aa, Aedes aegypti
LQEASEAYLVGLFEDT

SEQ ID NO: 29 - protein, 16 aa, Drosophila melanogaster
LQEASEAYLVGLFEDT

SEQ ID NO: 30 - protein, 16 aa, Bombyx mori
LQEASEAYLVGLFEDT

SEQ ID NO: 31 - protein, 16 aa, Caenorhabditis elegans
LQEAAEAYLVGLFEDT

SEQ ID NO: 32 - protein, 16 aa, Saccharomyces cerevisiae
LQESVEAYLVSLFEDT